(19) United States
(12) Patent Application Publication
(10) Pub. No.: US 2005/0163876 A1
(43) Pub. Date: Jul. 28, 2005

(54) Cancer treatment using natural plant products or essential oils or components from some pistacia species

(75) Inventor: Maria Paola Belloni Regazzo, Padova (IT)

Correspondence Address: MODIANO & ASSOCIATI S.p.A., Via Meravigli, 16, Milano, 20123, IT

(73) Assignee: DATA MEDICA PADOVA S.p.A.

(21) Appl. No.: 11/090,013

(22) Filed: Mar. 28, 2005

Related U.S. Application Data
(63) Continuation-in-part of application No. 10/676,101, filed on Oct. 2, 2003.

(51) Int. Cl. A61K 35/78

(52) U.S. Cl. 424/769

(57) ABSTRACT

Natural products or pharmaceutical compositions containing plant essential oil, from P. terebinthus, P. lentiscts, P. vera, P. integerrima or other Pistacia species, or their components, natural or synthetic, or mixture or derivatives thereof, for the prevention and treatment of cancer.

This is a Continuation-In-Part Application of U.S. Ser. No. 10/676,101 filed Oct. 2, 2003

### FIELD OF THE INVENTION

The present invention relates, in general, to therapeutically effective natural products and pharmaceutical compositions containing plant essential oil from Pistacia species, or components, for the prevention or the treatment of cancer in mammals, including humans.

### BACKGROUND OF THE INVENTION

It is known that nature represents a large source of therapeutically active drugs (Buffoni 1996). Indeed, the use of plants and other natural products for therapeutic purposes dates back basically to the beginning of humanity (Farnsworth 1985; Cragg. 1997).

Plants, like everything else, can be considered a mixture of different substances which despite a diversified metabolic fate, are maintained in a perfect equilibrium. Various drugs contain active principles with various pharmacologic activities, some of which are fundamental for some specific therapeutic use. Phytotherapy shouldn't thus be considered an alternative cure, but rather an important sector of pharmeacotherapy.

In fact, the phytotherapeutical remedies are often associate to the drugs of synthesis which, in various cases, are only to completion of therapies “natural”.

The natural products represented for time an excellent source of medicinal for the care of the cancer. The anti-tumorals of natural origin which are used successfully in the clinical practice are therefore several and some of these also very well known such as, for example, taxol, isolated from Taxus brevifolia; vincristine and the vinblastine, isolated from Vinca rosea; etoposide and teniposide, semisynthetic-derivatives of podophyllotoxin, isolated from Podophyllum peltatum. Natural products, moreover, given their structural variety, continue to attract interest in the antitumoral field (Farnsworth, 1990; Cragg 1999).

Among plant natural products having potential pharmacological activity, essential oils, pure mixtures of organic substances, play a central role. Even in the same species the composition of an essential oil is very variable due to the plant high sensitivity to different climatic conditions. Essential oils are generally obtained by compression or hydro-distillation. The distillation in steam current is the most widely used extraction method. Given the very complex composition of essential oils and the many quantitative changes occurring during the vegetative cycle of the plant, their characterization is quite difficult. The Pistacia genus (Anacardaceae) includes several species and is constituted by bushes or small trees, shrubs with resinous cortex.

The species found in the Mediterranean area are:
P. vera
P. terebinthus
P. lentiscus

The oil obtained by the Pistacia Vera nuts is scarcely used and is present only in a limited number of pharmaceutical preparations, while, essential oil extracted from the resin of Pistacia terebinthus has been shown to exert a significant anti-inflammatory activity in an experimental model of auricular inflammation in the rat (Giner-Larza 2000). Certainly much more used is instead the drug called rubber or mastic of lentiscus extracted by the Pistacia lentiscus var. chia. This is used in pills as expectorant, while its tonic and astringent action is exploited for the care of children's diarrheas. Mastic is also chewed for its light antiseptic oral activity and sometimes it is associated to camphor, sandracca and peruvian balm to eliminate bad breath. The finding of the lentiscus resin in mummies dating back to the seventh century A.C. shows that the Egyptians used this substance to embalm the dead exploiting its antiseptic activity (Colombini 2000).

It is also known that:
the water extract of Pistacia lentiscus, rich of potassium, sodium and magnesium, induces in the rat a hypotensive activity (Sanz 1987, 1988), probably due to the presence of n-butanolic and ethyl in the extract;
the essential oil obtained from the hydrodistillation of the mastic resin (gum) of P. lentiscus var. chia exerts an antibacteric activity in vitro more marked toward the gram+, respect to gram− bacteria. Identifying, in the essential oil, a “natural”antibacteric action is of interest also because it could replace preserving substances, often suspected of toxicity, cancerogenity and theratogenity (Magiatis 1999);
among the various extracts obtained by freeze-dried P. lentiscus leaves, the decoction is the only one to have in vitro, a good antibacteric activity in cultures of Staphylococcus aureus, Sarcina lutea and Escherichia coli and have a modest antimycotic activity, proven on cellular Torulapsis glabrata and Candida Parapsilosis coltures;

the mastic resin (gum) of the P. lentiscus var. chia, even when used at low dosages, acts quickly against peptic ulcer, thanks to its effectiveness against Helicobacter Pylori (Huwez 1998, Marone 2001).
the cortex and leaves from Pistacia lentiscus are used against diarrhea and gonorrhea. Essential oils from the Pistacia genus are rich in monoterpenes, which in fact represent the major components. Monoterpenes are non-nutritive dietary components also found in the essentials oils of many edible plants such as citrus, cherry, spearmint, dill, caraway, and others. Their natural functions may be as chemoattractants or chemorepellents, as they are largely responsible for the plant's pleasant fragrance. These simple 10 carbon isoprenoids are derived from the mevalonate pathway in plants but are not produced in mammals. For example, in spearmint and other plants, d-limonene is formed by the cyclization of geranylpyrophosphate by the enzyme limonene synthase (Croteau 1987). Limonene then serves as a precursor for other plant monocyclic monoterpenes such as carvone, carveol, and perillyl alcohol (Elson 1994).

The antitumor effects of dietary monoterpenes are attained with little or no host toxicity (Elson 1994, Crowell 1994 a, b, Evans 1995). A number of dietary monoterpenes have antitumor activity, exhibiting not only the ability to prevent the formation or progression of cancer, but to regress existing malignant tumors. Limonene and perillyl alcohol have well established chemopreventive activity against many cancer types. Indeed, d-limonene has a broad range of antitumor activities (Elson 1994, Crowell 1994). Dietary limonene reduces the incidence of spontaneous lymphomas in p53^{-/-} mice (Hursting 1995). Limonene, besides, has chemopreventive activity against spontaneous and chemically-induced rodent mammary, skin, liver, lung, and fore-stomach cancers, as well as ras oncogene-induced rat mammary cancer (Gould 1994). Furthermore, when administered either in pure form or as orange peel oil (95% d-limonene), limonene inhibits the development of chemically induced rodent mammary (Elegbede 1984, Elson 1988, Maltzman 1989, Wattenberg 1983), skin (Elegbede 1986 a), liver (Dietrich 1991), lung and forestomach (Wattenberg 1989, 1991) cancers (reviewed in Crowell and Gould 1994, Elson and Yu 1994, Elson 1995). In rat mammary carcinogenesis models, the chemopreventive effects of limonene are evident during the initiation phase of 7-12-dimethylbenz[a]anthracene (DMBA)²—induced cancer (Elson 1988) and during the promotion phase of both DMBA—and nitrosomethylurea (NMU)—induced cancers (Elson 1988, Maltzman 1989). Kawamori et al. (1996) reported that the development of azoxymethane-induced aberrant crypt foci in the colon of rats was significantly reduced when they were given 0.5% limonene in the drinking water. A Phase I clinical trial testing limonene's cancer chemotherapeutic activity is in progress (McNamee 1993).

Caraway seed oil, and its principal monoterpene, carvone, prevent chemically induced lung and forestomach carcinoma development when administered before the carcinogen (Wattenberg 1989). In addition, carveol (Crowell 1992) and menthol (Russin 1989) have chemopreventive activity against DMBA-induced rat mammary cancer when fed as 1% of the diet only during the initiation phase. Geraniol, an acyclic dietary monoterpene, has in vivo antitumor activity against murine leukemia, hepatoma and melanoma cells (Shoff 1991, Yu 1995) when administered before and after tumor cell transplantation.

In addition, many animal studies have shown perillyl alcohol to be a very powerful chemotherapeutics agent against several cancer types including pancreatic, breast, and liver cancer (Crowell 1999) and to have promotion phase chemopreventive activity against chemically induced liver cancer in rats (Mills 1995) and to be very effective at preventing tumor recurrences or secondary tumors in animals treated in a chemotherapy regimen (Haag 1994). Perillyl alcohol has chemotherapeutic activity against pancreatic cancer at doses that cause little toxicity to the host (Stark 1995). Perillyl alcohol reduced the growth of transplanted hamster pancreatic tumors to less than half that of controls. Moreover, a significant portion of perillyl alcohol-treated pancreatic tumors completely regressed, whereas none of the control tumors regressed (Stark 1995). Perillyl alcohol chemotherapy also reduces the growth rate of transplanted prostatic carcinomas in nude mice (Jeffers 1995). Thus, monoterpenes have chemotherapeutic activity against a number of solid types, including pancreatic cancer, one of the most refractory of all human cancers to available cancer therapies. The efficacy of perillyl alcohol chemotherapy against human cancer will be tested in forthcoming Phase I clinical trials (Phillips 1995)

Both limonene (Elegbede 1986 b, Haag 1992) and perillyl alcohol (Haag 1994) have chemotherapeutic activity against rat mammary tumors, causing the complete regression of >80% of established DMBA- or NMU-induced rat mammary tumors with limonene and the aromatase inhibitor 4-hydroxyandrostenedione was more effective than either drug alone.

Several mechanisms of action may account for the chemotherapeutic activities of monoterpenes. The blocking chemopreventive effects of limonene and other monoterpenes during the initiation phase of mammary carcinogenesis are likely due to the induction of Phase II carcinogen-metabolizing enzymes, resulting in carcinogen detoxificatoin. The post-initiation phase, tumor suppressive chemopreventive activity of monoterpenes may be due in part to the inhibition of isoprenylation of cell-growth associated small G proteins such as p21 ras by limonene, perillyl alcohol, and their metabolites (Crowell 1991, 1994). This inhibition occurs at the level of the prenyl-protein transferases. In addition, perillyl alcohol affects the mevalonate pathway by inhibiting ubiquinone biosynthesis as well as the conversion of lathosterol to cholesterol (Ren 1994). Chemotherapy of chemically-induced mammary tumors with monoterpenes results in tumor redifferentiation (Haag 1992). In limonene-treated mammary tumors, expression of the mannose-6-phosphate-insulin-like growth factor II receptor and transforming growth factor β1 are increased in the regressing, differentiating tumors, but not in the small number of tumors which are unresponsive to limonene (Jirtle 1993). In addition, the antitumor effects of dietary monoterpenes are attained with little or no host toxicity (Elson 1994, Crowell 1994, a,b, Evans 1995). In summary, a variety of dietary monoterpenes have been shown to be effective in the chemoprevention and chemotherapy of cancer. Now, monoterpenes research is progressing into human clinical trials for chemotherapeutic activity. Monoterpenes also possess many characteristics of ideal chemopreventive agents, namely, efficacious antitumor activity, commercial availability, low cost, oral bioavailability, low toxicity and novel mechanisms of action different from those of conventional cancer chemotherapeutic drugs, making it feasible to begin considering them for human cancer chemoprevention testing (Crowell 1996).

Several studies were led to the purpose to identify the chemical composition of the oil obtained by the leaves of P. lentiscus. Concluding that at second of the geographic origin area the various oils are characterized by a monoterpene unusual, the myrcene is present in particular by 19-25% in the oil coming from Spain and the Sicily (Calabro 1974, Boelens 1991) is possible from the analysis of the studies taken back in literature; the α-pinene is present for 16% in those French (Buil 1975); the terpen-4-ol is present by 22% in the one coming from the Sardinia (Castola 2000) and δ-3-carene the Egyptian oil characterizes (65%) (De Pooter 1991). Present members in less amount are a few sesquiterpeni, what: D-germacrene (9%) (Boelens 1991), the β-caryophyllene (3.5-9%) (Buil 1975, Boelens 1991), δ-cadinene and α-cadinolo (6% of everybody) (Buil 1975), the β-bisabolene, β-bourbonene and caryophyllene oxide (about 3-4% of everybody) (De Pooter 1991). The concentrations of the monoterpenes, besides, significantly change if the oil is obtained by the fruit. In particular, comparing two oils, the one coming from the Spain (Boelens 1991) and the one from the Australia (Wyllie 1990), it obtains, what component majors, respectively myrcene (72 and 39%), α-pinene (10 and 28%) and the limonene (87 and 11%).

The oils obtained for hydrodistillation by the mastic coming from Spain and Greece are instead characterized by a high α-pinene content (65-86%) and a low myrcene (3-25%) content (Scurbis 1975, Papageorgiou 1981; Katsiotis 1984; (Boelens 1991).

Of particular interest is the work of Migiatis and coll. (1999) who, using gas-cromatography and mass spectroscopy, identified 69 members of essential treols of P. lentiscus, var. chia, respectively obtained from the leaves, the twigs and the mastic.

### SUMMARY OF THE INVENTION

The present invention deals with the use of Pistacia natural products or essential oils and/or components, natural or synthetic, or mixtures or derivatives, and possibly other related natural products thereof for cancer prevention and treatment. In particular the present invention relates to the use of the above mentioned, by oral, parenteral and topic administration, also as adjuvant in combination with other cures, in preventive and therapeutic regimens directed towards the inhibition of cell growth or the killing of tumoral cells in humans and other animal species.

Additional objects and attendant advantages of the present invention will be set forth, in part, in the description that follows, or may be learned from practicing or using the present invention. The objects and advantages may be realized and attained by means of the features and combinations particularly recited in the appended claims. It is to be understood that the foregoing general description and the following detailed description provides the experimental basis for the invention, are exemplary and explanatory only and are not to be viewed as being restrictive of the invention, as claimed.

### DESCRIPTION OF THE DRAWINGS

FIG. 1 shows the cytotoxic effect of P. lentiscus essential oil from Portugal.

FIGS. 2a, 2b and 2c show the cytotoxic effect of single oil components.

FIG. 3 shows the cytotoxic effect of DM1C on MCF-7 cells.

FIG. 4 shows the cytotoxic effect of DM2A1 on 2008 and LoVo cells.

FIG. 5 shows the cytotoxic effect of DM3Z on MCF-7, 2008 and LoVo cells.

FIG. 6 shows the cytotoxic effect of DMF1 on MCF-7, 2008 and LoVo cells.

FIG. 7 shows the cytotoxic effect of D3MP1 on LoVo cells

FIG. 8 shows the cytotoxic effect of DMP on 2008 cells.

FIG. 9 shows the cytotoxic effect of DMG2 on LoVo cells.

FIG. 10 shows the cytotoxic effect of DM3P on LoVo cells.

FIG. 11 shows the cytotoxic effect of DM72 on MCF-7, 2008 and LoVo cells.

FIG. 12 shows the cytotoxic effect of DM2Z on 2008 cells.

FIG. 13 shows the cytotoxic effect of DM1Z on 2008 and LoVo cells.

FIG. 14 shows the cytotoxic effect of DMF3 on MCF-7, 2008 and LoVo cells.

FIG. 15 shows the cytotoxic effect of DMV2X on 2008 and LoVo cells.

FIG. 16 shows the cytotoxic effect of DMV5X on MCF-7, 2008 and LoVo cells.

FIG. 17 shows the cytotoxic effect of DM4a on LoVo cells.

FIG. 18 shows the cytotoxic effect of DM4P on 2008 and LoVo cells.

FIG. 19 shows the cytotoxic effect of DM5a on 2008 cells.

FIG. 20 shows the cytotoxic effect of DM4C on MCF-7, 2008 and LoVo cells.

FIG. 21 shows the cytotoxic effect of DMK on 2008 cells.

FIG. 22 shows the cytotoxic effect of DMF2 on MCF-7, 2008 and LoVo cells.

FIG. 23 shows the cytotoxic effect of DM1P on MCF-7 cells.

FIG. 24 shows the cytotoxic effect of DMG1 on MCF-7 and 2008 cells.

FIG. 25 shows the cytotoxic effect of DM2C on LoVo cells.

FIG. 26 shows the cytotoxic effect of DM3C on LoVo cells.

FIG. 27 shows the cytotoxic effect of DM2P on LoVo cells.

FIG. 28 shows the cytotoxic effect of DM1 S on MCF-7 cells.

FIG. 29 shows the cytotoxic effect of DMNP on 2008 and LoVo cells.

FIG. 30 shows the cytotoxic effect of DMFt2 on MCF-7, 2008 and LoVo cells.

FIG. 31 shows results of cytofluorimetric analysis.

FIG. 32 shows an evaluation of the nitrite concentration.

FIG. 33 shows the Pearson correlation and linear regression analyzes of the Para Cymene on LoVo cells.

FIG. 34 shows the Pearson correlation and linear regression analyzes of the Limonene on LoVo cells.

FIG. 35 shows the Pearson correlation and linear regression analyzes of the Endoborneol on LoVo cells.

FIG. 36 shows the Pearson correlation and linear regression analyzes of the Bornyl Acetate on LoVo cells.

FIG. 37 shows the Pearson correlation and linear regression analyzes of the Caryophyllene Oxide on LoVo cells.

### DETAILED DESCRIPTION OF THE INVENTION

The present invention regards a method for treating or preventing cancer in a mammal, including a human, comprising administrating an effective amount of a product obtainable from a plant of Pistacia genus with the exception of Pistacia lentiscus var. chia.

The present invention also regards a method for killing tumoral cells comprising exposing the tumoral cells to an effective amount of a product obtainable from a plant of Pistacia genus with the exception of Pistacia lentiscus var. chia.

“Products obtainable from a plant of the Pistacia genus” as used in the present specification and claims, means any part of a plant of Pistacia genus, as leaves, twigs, seeds, routs, nuts, galls, berries, branches, flowers, any natural product of a plant of Pistacia genus, as resins, products obtained from a plant of the Pistacia genus by any technique, for example but not limited to extraction, grinding, chemical, physical or chemical-physical treatments.

In one embodiment of the present invention, the products are products containing essential oils from plants of Pistacia genus, essential oils of plants of Pistacia genus as such or components thereof.

“Plant of Pistacia genus” as used in the present specification and claims means any plant of the Pistacia genus with the exception of P. lentiscus var. chia, of any geographical origin and of any species.

In one embodiment of the present invention, the plant of Pistacia genus is of European and Asiatic origin. In some embodiments of the present invention, the plant is of one of the species P. terebinthus, P. lentiscus, P. vera and P. integerrima.

The present invention particularly deals with the use of Pistacia natural products or essential oils and/or components, natural or synthetic, or mixtures or derivatives, and possibly other related natural products thereof for cancer prevention and treatment. In particular the present invention relates to the use of the above mentioned, by oral, parenteral and topic administration, also as adjuvant in combination with other cures, in preventive and therapeutic regimens directed towards the inhibition of cell growth or the killing of tumoral cells in humans and other animal species.

### BRIEF DESCRIPTION OF EXPERIMENTAL DATA

The features and advantages of the present invention will become more clearly appreciated from the following description of experimental data indicating the in vitro anti-tumoral activity of essential oils extracted from various species of Pistacia.

Methods:

Plant Collection:

Aerial parts (leaves and twigs, branches, fruits, nuts, seeds, flowers and galls) of the plants were collected in different seasons and at various times of the day in three different Italian regions: Veneto (P. terebinthus); Tuscany (P. lentiscus); Sicily (P. vera). Other samples were collected outside Italy: one of the P. lentiscus samples was collected in Portugal and Portuguese essential oil was obtained from John Steel (3949 Longridge Ave. Sherman Oaks—Calif.—USA), whereas one of the P. terebinthus samples (flowers) was collected in Provence (France); the P. integerrima plants were collected in Nepal and Nepalese essential oil was obtained from Bluebell Herbal Products (Bhaktapur—Nepal); the P. lentiscus var. chia plants were collected in Chios (Greece) and essential oil, derived from mastic resin (gum), was obtained from “Anemos” Benetos John—Galatoulas George Co (Chios—Karfas R D, Kontari, Chios, Greece).

Plant parts were generally collected and then rinsed, dried and frozen at −21° C. within three hours from collection. The material was then hydro-distilled within few months. The vegetable material exposed to hydro-distillation consisted in leaves, flowers, fruits, branches, galls and nuts of P. terebinthus, P. lentiscus, P. vera and P. integerrima. Samples were washed and dried carefully and preserved to low temperature (−21°) to keep unchanged their phytochemical composition until the moment of the distillation. All the samples, before being exposed to distillation, were minced to obtain the maximum extraction yield and make the process of diffusion of the essence easier (Boelens 1991, De Pooter 1991, Magiatis 1999, Papageorgiou 1981).

Essential Oil Extraction

The equipment used for the extraction of the essential oils consisted in a container in Inox steel (10lt), in which 1750 ml of distilled water are added, separated from the minced drug by a steel grid, to avoid direct contact of the drug with the extraction water. The sample is compacted by a further grill to avoid handling of the drug during the extraction proceeding and at the same time let the water steam freely flow down. To this point the steel container closed. The boiler is equipped with a thermometer, to be able to check at every moment of the distillation the temperature in boiler, and is also connected to a distillation column in steel, taking a coolant to the superior extremity, always in steel, with cooling running water. Water in the range container to the ebullition develops steam which going beyond the grill, laps the drug and extracts the essences contained, gone beyond the grill, the steam is directed along the distillation column and condensed in the coolant; the water mixture-essences to this point is collected in a graded cylinder containing some ethilic ether to dissolve the essential oil extracted by the steam, which for their liophylic nature, present a greater affinity for the solvent. The organic phase is then treated with natrium sulphate anhidrous, filtered and evaporated (Boelens 1991, De Pooter 1991, Magiatis 1999, Papageorgiou 1981). The distillation was usually led for up to 4 hours with constant heating; the mean initial weight of the sample was: leaves (450 g), branches (250 g), berries (100 g), flowers (270 g ). The yield for the various parts of the plant, express as percent of initial wet weight was: leaves (0,05%), branches (0,06%), berries (0,11%), flowers (0,08%), galls (0,40%).

Determination of Oil Chemical Composition

The chemical composition of the essential oils obtained by P. lentiscus, P. lentiscus var. chia, P. terebinthus, P. Vera and P. integerrima was determined by means of analysis gascromatographic coupled to a detector mass spectrophotometer (GC/MS), using an operating system Hewlett-Packard 6890-5973 in endowed modality EI (electronic ionization with potential 70 eV), equipped of capillary columne HP-5 MS (30 m×0.25 mm), with thickness of the equal film to 0.25 m, stationary phase of polidimetil silossano al 95%. It was operated applying one program of temperature starting from 60° C. for the first three minutes raising up to 280° with a speed of 3° C./min. for 5 minutes; the Injector was kept to 200° C. The spectrum obtained was compared with Wiley library's mass spectra (Boelens 1991, De Pooter 1991, Magiatis 1999, Papageorgiou 1981).

Biological Assays

Samples preparation: the stock solution of essential oils (9%) were prepared in DMSO (1%) and in culture medium (90%). All the procedures were carried out under sterile conditions. Before each experiment the stock solutions were diluted with growth medium and used immediately.

Cell Lines:

Cytotoxicity was evaluated on three human adenocarcinoma cell lines: ovarian (2008), breast (MCF-7), colon (LoVo).

The human ovarian adenocarcinoma cell line 2008, kindly supplied by Prof. G. Marverti (Department of Biomedical Sciences, University of Modena) and were maintained in RPMI 1640 medium supplemented with 10% heat-inactivated FCS (Foetal Calf Serum), 1% antibiotics (all products of Biochrom KG Seromed, Berlin), and 1% 2 mM glutamine (Merck).

The human breast adenocarcinoma cells lines MCF-7, supplied by the Experimental Zooprophylaxis Institute of the Lombardy and Emilia (Brescia, Italy), were cultured in MEM with Eagle's salts, plus 10% heat-inactivated Foetal Calf Serum, 1% antibiotics and sodium piruvate (all products of Biochrom KG Seromed), 1% 200 mM glutamine (Merck).

The human colon adenocarcinoma cell line LoVo, kindly supplied by Dr. G. Toffoli, Oncologic Reference Centre, Aviano, Italy. The cell line was cultured in Ham's F12 with the Addition of 10% heat inactivated Foetal Calf Serum, 1% glutamine 200 mM (Merk), 1% natrium piruvate (Seromed Biochrom KG, Berlin).

Cytotoxicity:

The cells (1×10⁵ cells/ml) were seeded in 96-well tissue plates (Falcon) and treated 24 h later with each essential oil at different concentrations. After 3 h exposure, medium was discarded, the plates were washed with sterile PBS and then added with growth medium.

The cytotoxic effect was evaluated was by tetrazolium salts reduction assay (MTT) after 21 h of incubation. An amount of 20 μL of MTT solution (5 mg/mL in PBS) was added to each well, and plates were incubated for 4 h at 37° C. DMSO (150 μL) was added to all wells and mixed thoroughly to dissolve the dark-blue crystals. The absorbance was measured on a microculture plate reader (Titertek Multiscan) using a test wavelength of 570 nm and a reference wavelength of 630 nm.

Statistical Analysis:

The IC₅₀ and the dose-response curve best-fit was determined and elaborated by Software “GraphPad Prism Version 3.0”. The IC₅₀ was determined also by linear regression analysis, after logit transformation (Rodbard 1975) using GraphPad Prism Version 3.0 Software, but data no shown. For each assay the experiments were performed in triplicate and each essential oil was tested in triplicate on three different cell lines.

The Pearson correlation and linear regression analyzes between the oil components and biological activity was also determined. To the scope all data were collected in a data base and analysed by the GraphPad Prism Version 3.0 statistical software to identify substances directly responsible of biological activity.

Nitrite Assay:

The nitrite concentration in the culture medium was measured as an indicator of NO production using Griess reaction. One hundred microliters of each supernatant was mixed with the same volume of Griess A reagent (1% sulphanilamide in 5% phosphoric acid) and after 10 minutes 100 ml of Griess B reagent (0.1% naphthylethylenediamine dihydrochloride in water) was added. After 15 minutes the absorbance of mixture was determined at 543 nm.

Cytofluorimetry: cells are collected and rinsed twice with cold PBS and then resuspended in 1× binding buffer at a concentration of 1×106 cells/ml. Transfer 100 μl of the solution (1×105 cells) to a 5 ml culture tube. 5 μl of Annexin V-FITC and 5 μl of PI are added. Cells are gently vortexed and incubated for 15 min at RT (25° C.) in the dark. 400 μl of 1× binding buffer to each tube are then added and samples analyzed by flow cytometry within one hour. Annexin V is a 35-36 kDa Ca2+ dependent phospholipid-binding protein that has a high affinity for PS, and binds to cells with exposed PS. Annexin V may be conjugated to fluorochromes such as Propidium Iodide (PI). This format retains its high affinity for PS and thus serves as a sensitive probe for flow cytometric analysis of cells that are undergoing apoptosis. Since externalization of PS occurs in the earlier stages of apoptosis, Annexin V-FITC staining can identify apoptosis at an earlier stage than assays based on nuclear changes such as DNA fragmentation. Annexin V-FITC staining precedes the loss of membrane integrity which accompanies the latest stages of cell death resulting from either apoptotic or necrotic processes. Therefore, staining with Annexin V-FITC is typically used in conjunction with a vital dye such as Propidium Iodide to allow the investigator to identify early apoptotic cells (Annexin V-FITC positive, PI negative). For example, cells that are viable are Annexin V-FITC and PI negative; cells that are in early apoptosis are Annexin V-FITC positive and PI negative; and cells that are in late apoptosis or already dead are both Annexin V-FITC and PI positive. This assay does not distinguish, per se, between cells that have already undergone apoptotic death and those that have died as a result of a necrotic pathway because in either case, the dead cells will stain with both Annexin-FITC and PI. However, when apoptosis is measured over time, cells can be often tracked from Annexin V-FITC and PI negative (viable, or no measurable apoptosis), to Annexin V-FITC positive and PI negative (early apoptosis, membrane integrity is present) and finally to Annexin V-FITC and PI positive (end stage apoptosis and death). The movement of cells through these three stages suggests apoptosis. In contrast, a single observation indicating that cells are both Annexin V-FITC and PI positive, in of itself, reveals less information about the process by which the cells underwent their demise.

Reagents

1. Annexin V-FITC

2. Propidium Iodide.

3. 10× Annexin V Binding Buffer.

Staining

1. Wash cells twice with cold PBS and then resuspend cells in 1× binding buffer at a concentration of 1×106 cells/ml.

2. Transfer 100 μl of the solution (1×105 cells) to a 5 ml culture tube.

3. Add 5 μl of Annexin V-FITC and 5 μl of PI.

4. Gently vortex the cells and incubate for 15 min at RT (25° C.) in the dark.

5. Add 400 μl of 1× binding buffer to each tube. Analyze by flow cytometry within one hour.

The results are shown in Table 1a, Table 1b and Table 1c.

Other Materials

Single components of essential oils (Bomyl acetate, d-Limonene, α-Pinene, β-Myrcene and Para Cymene) were obtained from SIGMA srl.

TABLE 1a
P. lentiscus Composition

| | | Leaves | | | | | | Berries | | | Branches | |
|---|---|---|---|---|---|---|---|---|---|---|---|---|
| RT* | Oil Composition | Portuguese Oil | DM1C | DM2A1 | DM3Z | DMF1 | DM3P1 | DMP | DMG2 | DM3P | DM72 | DM2Z |
| 6.08 | Tricyclene | — | 0.33 | 0.58 | 0.14 | 0.06 | 1.20 | — | — | — | 0.90 | — |
| 6.22 | α-Phellandrene | — | 1.51 | 0.12 | 0.14 | 0.20 | 0.15 | 0.61 | — | 0.20 | — | 0.14 |
| 6.48 | α-Pinene | 12.95 | 11.45 | 12.97 | 9.17 | 24.68 | 12.86 | 20.13 | 6.11 | 40.68 | 18.15 | 13.73 |
| 6.94 | Camphene | 2.05 | 1.46 | 2.49 | 0.52 | 0.58 | 4.96 | 1.06 | — | 0.98 | 3.90 | 0.22 |
| 7.78 | Sabinene | 1.16 | 4.31 | 4.56 | 2.87 | 2.32 | 4.47 | 0.99 | 1.21 | 0.86 | 2.83 | 6.77 |
| 7.88 | β-Pinene | 4.33 | 3.07 | 4.38 | 1.15 | 8.64 | 4.25 | 4.13 | 0.48 | 7.60 | 4.19 | 0.94 |
| 8.40 | β-Myrcene | 8.42 | 0.97 | 0.67 | 4.45 | 0.91 | 9.23 | 23.80 | 57.79 | 1.08 | 4.08 | 5.47 |
| 8.86 | 1-Phellandrene | 0.69 | 4.64 | 0.80 | 4.60 | 2.12 | 2.07 | 3.84 | 3.79 | 0.92 | 0.81 | 8.35 |
| 9.02 | Methylanisol | — | — | — | — | — | — | — | — | — | — | — |
| 9.07 | δ-Carene | 0.67 | — | — | — | — | — | — | — | — | 3.14 | — |
| 9.33 | α-Terpinene | 0.56 | 4.66 | 3.60 | 3.76 | 3.11 | 2.82 | 2.71 | 0.90 | 1.13 | 0.72 | 1.73 |
| 9.69 | Para Cymene | 6.43 | 0.64 | 0.22 | 0.50 | 0.35 | 0.30 | 0.48 | 0.34 | 0.16 | 0.42 | 0.98 |
| 9.81 | β-Phellandrene | — | 6.48 | 4.93 | 5.79 | 11.35 | 4.73 | 6.48 | 3.44 | 8.03 | — | 6.61 |
| 9.89 | Limonene | 17.55 | — | — | — | — | — | — | — | — | — | — |
| 10.21 | Cis-Ocimene | — | 0.60 | 0.35 | 1.00 | 0.72 | 0.52 | — | 0.29 | 0.22 | 1.08 | 0.61 |
| 10.62 | Trans-β-Ocimene | 0.14 | 0.41 | 0.23 | 0.53 | 0.46 | 0.35 | — | 0.57 | — | 0.32 | 0.23 |
| 10.96 | Butanoic Acid | — | — | — | — | 0.55 | — | — | — | 1.07 | — | — |
| 11.03 | γ-Terpinene | 1.24 | 7.42 | 5.90 | 6.11 | 4.89 | 4.94 | 4.93 | 1.85 | 2.24 | 1.51 | 2.96 |
| 12.26 | α-Terpinolene | 0.61 | 2.48 | 2.08 | 2.35 | 2.59 | 1.75 | 2.08 | 0.63 | 2.27 | 0.64 | 1.04 |
| 12.49 | 2-Nonanone | — | 0.34 | 0.17 | 0.19 | 0.63 | 0.30 | 0.44 | 0.31 | 1.65 | — | — |
| 12.60 | α-Pinene Oxide | — | — | — | — | — | — | — | — | — | — | — |
| 12.74 | Linalool | 0.11 | — | 0.15 | 0.22 | 0.39 | — | — | — | — | — | 0.36 |
| 12.96 | Pinocarveol | — | — | — | — | — | — | — | — | — | — | — |
| 14.64 | Pinocarvone | — | — | — | — | — | — | — | — | — | — | — |
| 14.70 | Camphor | 0.13 | — | — | — | 0.08 | 0.31 | — | — | — | — | — |
| 15.74 | Endoborneol | 6.77 | 0.12 | 0.34 | — | 0.24 | 0.43 | 0.32 | — | 0.65 | — | — |
| 16.20 | 1-4 Terpineol | 3.37 | 14.89 | 12.26 | 13.11 | 7.12 | 10.43 | 8.78 | 4.09 | 2.63 | 3.03 | 6.98 |
| 16.79 | α-Terpineol | 0.78 | 4.92 | 5.36 | 6.42 | 7.41 | 4.96 | 7.97 | 1.11 | 9.83 | 1.41 | 1.13 |
| 17.48 | Myrtenol | — | — | — | — | — | — | — | — | — | — | — |
| 19.44 | Octanoid Acid | — | 0.18 | 0.13 | 0.22 | — | 0.19 | — | — | 0.54 | — | 0.17 |
| 21.04 | Bornyl Acetate | 24.48 | 2.12 | 1.77 | 0.17 | — | 0.52 | 1.94 | — | — | 0.98 | 0.28 |
| 21.39 | Verbenone | — | — | — | — | — | — | — | — | — | — | — |
| 21.42 | 2-Undecanone | 0.30 | 0.69 | 0.19 | 0.55 | 0.99 | 0.67 | 0.41 | 0.59 | 1.38 | 1.47 | 0.65 |
| 24.77 | α-Ylangene | — | 0.42 | 0.87 | 0.70 | — | 0.30 | — | 0.32 | — | 0.46 | 0.60 |
| 25.39 | β-Cubebene | — | — | 0.25 | 0.21 | — | — | — | — | — | — | 0.34 |
| 25.47 | β-Elemene | — | 0.15 | 0.19 | 0.23 | 0.11 | 0.16 | — | — | — | 0.52 | 0.51 |
| 26.61 | β-Caryophyllene | 2.09 | 2.79 | 3.21 | 3.24 | 4.02 | 4.92 | 0.44 | 0.81 | 1.18 | 3.91 | 1.70 |
| 27.85 | α-Cubebene | — | — | 0.05 | 0.24 | — | — | — | — | — | — | — |
| 27.96 | α-Humulene | 0.17 | 1.02 | 0.88 | 1.13 | 0.48 | 1.09 | 0.25 | 0.37 | — | 1.10 | 0.60 |
| 28.25 | Alloaromandrene | 0.10 | 0.30 | 0.34 | 0.39 | — | 0.24 | — | — | — | 0.44 | 0.48 |
| 28.99 | α-Amorphene | 0.37 | 0.85 | 1.51 | 1.30 | 0.17 | 0.64 | 0.35 | 0.80 | 0.39 | 1.16 | 1.13 |
| 29.10 | Germacrene D | 0.25 | 4.71 | 6.82 | 5.00 | 1.37 | 4.34 | 2.06 | 2.76 | 1.23 | 8.36 | 8.32 |
| 29.66 | α-Elemene | — | 0.33 | 0.41 | 0.51 | 0.26 | 0.34 | — | — | 0.35 | 0.90 | 0.99 |
| 29.89 | α-Muurolene | 0.13 | 0.71 | 1.21 | 1.07 | 0.33 | 0.68 | — | 0.68 | 0.44 | 0.98 | 0.93 |
| 30.05 | Germacrene A | — | 0.14 | 0.18 | 0.30 | 0.16 | 0.23 | — | — | — | 0.44 | 0.68 |
| 30.42 | Butylated Hydroxy Toluene | — | 0.60 | 0.81 | 1.36 | 0.44 | 1.14 | 0.88 | 1.23 | 1.72 | 4.33 | 1.25 |
| 30.81 | δ-Cadinene | 0.20 | 2.82 | 4.80 | 4.40 | 1.11 | 2.52 | 1.32 | 3.28 | 1.74 | 4.44 | 3.99 |
| 32.26 | Spathulenol | — | — | — | — | — | — | — | — | — | — | — |
| 32.78 | γ-Cadinene | 0.54 | — | — | — | — | — | — | — | — | 0.19 | 0.87 |
| 33.20 | Caryophyllene Oxide | 2.53 | — | — | — | 0.12 | — | — | — | — | 0.83 | — |
| 34.79 | Cadina 1,4-Diene | — | 0.83 | 1.26 | 1.50 | 0.20 | 0.61 | — | 0.46 | — | 0.86 | 0.71 |
| 34.92 | γ-Eudesmol | — | 1.21 | 0.96 | 1.07 | 0.48 | 0.77 | 0.41 | 0.63 | 0.28 | — | 0.61 |
| 35.31 | α-Cadinol | — | 2.50 | 3.29 | 4.13 | 1.63 | 2.52 | 0.76 | 1.96 | 0.97 | 3.18 | 2.70 |
| 35.45 | α-Copaene | — | 0.75 | 1.03 | 1.29 | 0.49 | 0.76 | — | 0.53 | 0.31 | 1.22 | 0.81 |
| 35.58 | β-Eudesmol | — | 0.47 | 0.31 | 0.41 | 0.19 | 0.28 | — | — | 0.20 | — | 0.46 |
| 35.80 | T-Cadinolo | 0.85 | 3.16 | 3.90 | 4.73 | 2.61 | 3.55 | 1.15 | 2.79 | 1.68 | 4.53 | 3.77 |
| 56.28 | Nonadecane | — | — | — | — | — | — | — | — | — | — | — |
| 61.56 | Octadecane | — | — | — | — | — | — | — | — | — | — | — |
| 67.79 | 4Chloro2Phenylaniline | — | — | — | — | — | — | — | — | — | — | — |
| 67.96 | Benzene1Methoxy2,3,5Trimet | — | — | — | — | — | — | — | — | — | — | — |
| 69.78 | 4,12BisHydroxymethyl | — | — | — | — | — | — | — | — | — | — | — |

TABLE 1b
P. terebinthus Composition

| | | Leaves | | | | | | | | | Fruits | | Galls | | Flowers | | | Branches |
|---|---|---|---|---|---|---|---|---|---|---|---|---|---|---|---|---|---|---|
| RT* | Oil Composition | DM1Z | DM2S | DMF3 | DMV2X | DMV5X | DM4a | DM4p | DM5a | DM4C | DMK | DMF2 | DMG1 | DM1P | DM2C | DM3C | DM3A | DM2P |
| 6.08 | Tricyclene | — | — | — | — | — | 0.25 | — | — | — | 0.30 | — | — | — | 0.48 | 0.45 | — | 0.18 |
| 6.22 | α-Phellandrene | — | — | — | — | — | — | — | — | — | — | — | — | — | 0.21 | — | — | 0.36 |
| 6.48 | α-Pinene | 35.63 | 12.17 | 41.03 | 11.61 | 14.09 | 60.99 | 10.04 | 10.99 | 18.67 | 31.63 | 8.69 | 54.19 | 42.16 | 28.90 | 26.43 | 11.74 | 28.72 |
| 6.94 | Camphene | 0.55 | 0.26 | 1.02 | — | — | 1.56 | 0.24 | 0.37 | 0.46 | 1.50 | 0.48 | 0.69 | 0.67 | 2.12 | 1.83 | — | 1.06 |
| 7.78 | Sabinene | — | 0.62 | — | 0.39 | — | 0.22 | 0.15 | 0.28 | — | 1.20 | 0.85 | 0.65 | 0.18 | 2.00 | 0.91 | 1.41 | 4.53 |
| 7.88 | β-Pinene | 1.12 | 1.14 | 6.17 | 1.39 | 0.71 | 1.87 | 0.32 | 1.48 | 3.27 | 7.44 | 1.39 | 3.68 | 1.81 | 6.40 | 5.85 | 2.12 | 4.07 |
| 8.40 | β-Myrcene | 1.57 | 1.68 | 1.37 | 1.82 | 2.03 | 1.98 | 1.41 | 1.60 | 2.90 | 1.21 | 1.11 | 1.05 | 1.42 | 1.68 | 0.97 | 31.56 | 1.67 |
| 8.86 | 1-Phellandrene | 0.62 | 0.35 | 0.49 | 0.75 | 0.62 | 0.47 | 0.19 | 0.29 | 0.27 | 3.34 | 7.66 | 0.49 | 6.61 | 0.32 | 0.37 | 0.61 | 0.59 |
| 9.02 | Methylanisol | — | — | — | — | — | — | — | — | — | — | — | — | — | — | — | — | — |
| 9.07 | δ-Carene | — | — | — | 1.23 | — | 0.76 | — | — | — | — | — | — | — | — | — | — | — |
| 9.33 | α-Terpinene | — | 0.43 | 0.83 | 0.71 | — | 0.44 | — | 0.21 | 0.34 | 0.25 | 0.34 | — | — | 0.65 | 0.51 | — | 1.46 |
| 9.69 | Para Cymene | — | 0.11 | — | — | — | 0.17 | — | — | — | 0.38 | — | — | 0.18 | 0.20 | — | 0.79 | 0.49 |
| 9.81 | β-Phellandrene | — | — | — | — | — | — | — | — | — | — | — | — | — | — | — | — | — |
| 9.89 | Limonene | 27.45 | 50.70 | 6.09 | 21.04 | 60.47 | 6.16 | 1.23 | 8.03 | 60.24 | 12.59 | 32.85 | 13.97 | 31.07 | 28.76 | 23.03 | 5.58 | 6.56 |
| 10.21 | Cis-Ocimene | 19.63 | 15.64 | 10.93 | 20.97 | 3.86 | 6.31 | 63.60 | 54.19 | 0.96 | 6.79 | 17.99 | 1.89 | 0.37 | — | 3.88 | — | 13.44 |
| 10.62 | Trans-β-Ocimene | 5.49 | 4.45 | 3.80 | 6.04 | 1.17 | 1.86 | 18.29 | 16.46 | 0.38 | 1.66 | 5.01 | 0.40 | — | — | 1.22 | — | 3.33 |
| 10.96 | Butanoic Acid | — | — | — | — | — | — | — | — | — | — | — | — | — | — | — | — | — |
| 11.03 | γ-Terpinene | 0.22 | 0.69 | 0.48 | 0.62 | — | 0.49 | 0.17 | 0.34 | 0.60 | 0.33 | 0.42 | 0.12 | — | 1.08 | 0.77 | 0.46 | 2.46 |
| 12.26 | α-Terpinolene | 0.92 | 0.93 | 17.07 | 16.59 | 4.90 | 7.13 | 0.38 | 0.46 | 1.76 | 0.44 | 1.27 | 0.26 | 0.28 | 0.57 | 2.99 | — | 1.08 |
| 12.49 | 2-Nonanone | — | — | — | — | — | — | — | — | — | — | — | — | — | — | — | — | — |
| 12.60 | α-Pinene Oxide | — | — | — | — | — | — | — | — | — | — | — | — | — | — | — | — | — |
| 12.80 | Linalool | — | — | — | — | — | 0.18 | — | — | — | — | 0.17 | — | — | — | — | — | — |
| 12.96 | Pinocarveol | — | — | — | — | — | — | — | — | — | — | — | — | — | — | — | — | — |
| 14.64 | Pinocarvone | — | — | — | — | — | — | — | — | — | — | — | — | — | — | — | — | — |
| 14.70 | Camphor | — | — | — | — | — | — | — | — | — | — | — | — | — | — | — | — | — |
| 15.74 | Endoborneol | — | — | — | — | — | — | — | — | — | — | — | — | — | — | — | — | — |
| 16.20 | I-4 Terpineol | 0.31 | 1.63 | 0.29 | 1.86 | 1.21 | 0.29 | 0.35 | 0.50 | 0.46 | 1.00 | 0.87 | 0.18 | 0.13 | 2.86 | 1.55 | — | 8.91 |
| 16.79 | α-Terpineol | 1.63 | 4.05 | 5.22 | 1.92 | 1.18 | 2.23 | 1.35 | 1.22 | 2.87 | 2.43 | 0.93 | 0.35 | 1.36 | 1.69 | 1.45 | — | 2.26 |
| 17.48 | Myrtenol | — | — | — | — | — | — | — | — | — | — | — | — | — | — | — | — | — |
| 19.44 | Octanoid Acid | — | — | — | — | — | — | — | — | — | — | — | — | — | — | — | — | — |
| 21.04 | Bornyl Acetate | — | — | 0.47 | — | — | — | — | — | — | 0.64 | 0.35 | — | 0.18 | — | — | — | 0.23 |
| 21.39 | Verbenone | — | — | — | — | — | — | — | — | — | — | — | — | — | — | — | — | — |
| 21.42 | 2-Undecanone | — | — | — | — | — | — | — | — | — | — | — | — | — | — | — | — | — |
| 24.77 | α-Ylangene | 0.23 | 0.16 | — | — | — | — | — | — | — | 4.51 | 0.96 | 0.15 | 0.19 | — | 2.14 | — | 0.51 |
| 25.39 | β-Cubebene | — | — | — | — | — | — | — | — | — | 1.60 | 0.40 | — | — | — | 0.37 | — | 0.19 |
| 25.47 | β-Elemene | — | — | — | — | — | — | — | — | — | — | — | — | — | — | — | — | — |
| 26.61 | β-Caryophyllene | 3.23 | 1.38 | 2.75 | 4.72 | 2.00 | 3.86 | 0.62 | 1.29 | 3.43 | 4.77 | 1.64 | 14.33 | 0.94 | 0.97 | 8.05 | 0.63 | 1.06 |
| 27.85 | α-Cubebene | — | — | — | — | — | — | — | — | — | 0.26 | 0.42 | — | — | — | — | — | 0.74 |
| 27.96 | α-Humulene | 0.47 | 0.33 | — | 0.86 | 1.07 | 1.21 | — | — | 1.46 | 1.47 | 0.55 | 2.15 | 0.33 | 0.65 | 1.24 | — | 0.22 |
| 28.25 | Alloaromandrene | — | — | — | — | — | — | — | — | — | — | — | — | — | — | — | — | — |
| 28.99 | α-Amorphene | — | 0.24 | — | — | — | — | 0.17 | — | 0.30 | 0.55 | 0.24 | — | 0.15 | 0.43 | 0.48 | 1.27 | 0.77 |
| 29.10 | Germacrene D | 0.23 | 0.62 | — | 1.56 | 1.40 | — | 0.14 | 0.45 | — | 2.24 | 9.42 | 2.91 | 7.08 | 5.87 | 2.44 | 4.45 | 2.91 |
| 29.66 | α-Elemene | — | — | — | — | — | — | — | — | — | 0.18 | — | — | — | — | — | — | 0.46 |
| 29.89 | α-Muurolene | — | — | — | — | — | — | — | — | — | 0.25 | — | — | — | 0.33 | 0.27 | 0.76 | 0.47 |
| 30.05 | Germacrene A | — | — | — | — | — | — | — | — | — | — | — | — | 0.14 | — | — | — | — |
| 30.42 | Butylated Hydroxy Toluene | 0.32 | 0.77 | 0.37 | 3.34 | 2.36 | 0.62 | 0.46 | 0.45 | 0.55 | 0.58 | 0.46 | 0.30 | 0.45 | 1.56 | 1.58 | — | 1.82 |
| 30.81 | δ-Cadinene | 0.44 | 0.67 | — | 1.32 | 0.92 | 0.31 | 0.31 | 0.63 | 0.61 | 6.42 | 2.33 | 0.31 | 0.55 | 1.22 | 3.61 | 3.06 | 3.51 |
| 32.26 | Spathulenol | — | — | — | — | — | — | — | — | — | — | — | — | — | — | — | — | — |
| 32.78 | γ-Cadinene | — | — | — | — | — | — | — | — | — | — | — | — | — | — | — | 0.84 | — |
| 33.20 | Caryophyllene Oxide | — | 0.23 | — | — | — | 0.27 | — | — | — | 0.40 | — | 0.39 | — | — | 0.75 | — | — |
| 34.79 | Cadina 1,4-Diene | — | — | — | — | — | — | — | — | — | 0.31 | 0.17 | — | — | — | 0.36 | — | 0.31 |
| 34.92 | γ-Eudesmol | — | — | — | — | — | — | — | — | — | — | — | — | 0.70 | — | — | — | — |
| 35.31 | α-Cadinol | — | — | — | — | — | — | — | — | — | 0.26 | — | — | 0.21 | 0.81 | 0.73 | — | 0.53 |
| 35.45 | α-Copaene | — | — | — | — | — | — | — | — | — | 0.39 | 0.55 | — | — | 0.25 | 0.19 | — | — |
| 35.58 | β-Eudesmol | — | — | — | — | — | — | — | — | — | — | — | — | 0.21 | — | — | — | — |
| 35.80 | T-Cadinolo | — | 0.17 | — | — | — | — | — | — | — | 0.31 | 0.65 | — | 0.30 | 1.14 | 0.87 | 1.26 | 0.36 |
| 56.28 | Nonadecane | — | — | — | — | — | — | — | — | — | — | — | — | — | 2.20 | 1.04 | — | — |
| 61.56 | Octadecane | — | — | — | 0.47 | — | — | — | — | 0.25 | — | — | — | — | 1.99 | 0.71 | — | — |
| 67.79 | 4Chloro2Phenylaniline | — | — | — | — | — | — | — | — | — | — | — | — | — | — | — | — | — |
| 67.96 | Benzene1Methoxy2,3,5Trimet | — | — | — | — | — | — | — | — | — | — | — | — | — | — | — | — | — |
| 69.78 | 4,12BisHydroxymethyl | — | — | — | — | — | — | — | — | — | — | — | — | — | — | — | — | — |

TABLE 1C
P. Vera and P. integerrima Composition

| | | P. vera | P. integerrima | |
|---|---|---|---|---|
| | | Nuts | Galls | |
| RT* | Oil Composition | DM1S | DMNP | DMFt2 |
| 6.08 | Tricyclene | — | — | — |
| 6.22 | α-Phellandrene | — | — | — |
| 6.48 | α-Pinene | 0.99 | 14.41 | 22.37 |
| 6.94 | Camphene | — | 0.94 | 1.32 |
| 7.78 | Sabinene | — | 2.51 | 2.46 |
| 7.88 | β-Pinene | — | 5.62 | 7.63 |
| 8.40 | β-Myrcene | — | 2.81 | 3.20 |
| 8.86 | 1-Phellandrene | — | 0.56 | 0.77 |
| 9.02 | Methylanisol | — | — | — |
| 9.07 | δ-Carene | — | 4.06 | 8.36 |
| 9.33 | α-Terpinene | — | 0.80 | 1.80 |
| 9.69 | Para Cymene | — | 2.09 | 3.72 |
| 9.81 | β-Phellandrene | — | — | — |
| 9.89 | Limonene | 1.54 | 5.94 | 7.81 |
| 10.21 | Cis-Ocimene | — | — | — |
| 10.62 | Trans-β-Ocimene | — | — | — |
| 10.96 | Butanoic Acid | — | — | — |
| 11.03 | γ-Terpinene | — | 1.52 | 3.74 |
| 12.26 | α-Terpinolene | — | 0.57 | 1.61 |
| 12.49 | 2-Nonanone | — | — | — |
| 12.60 | α-Pinene Oxide | — | — | — |
| 12.80 | Linalool | — | — | — |
| 12.96 | Pinocarveol | — | — | — |
| 14.64 | Pinocarvone | — | — | — |
| 14.70 | Camphor | — | — | — |
| 15.74 | Endoborneol | — | 3.12 | 1.02 |
| 16.20 | 1-4 Terpineol | 2.13 | 29.52 | 20.69 |
| 16.79 | α-Terpineol | 2.56 | 14.08 | 8.74 |
| 17.48 | Myrtenol | — | — | — |
| 19.44 | Octanoid Acid | — | — | — |
| 21.04 | Bornyl Acetate | — | 4.55 | 1.55 |
| 21.39 | Verbenone | — | — | — |
| 21.42 | 2-Undecanone | — | — | — |
| 24.77 | α-Ylangene | — | — | — |
| 25.39 | β-Cubebene | — | — | — |
| 25.47 | β-Elemene | — | — | — |
| 26.61 | β-Caryophyllene | 0.60 | 2.81 | 1.96 |
| 27.85 | α-Cubebene | — | — | — |
| 27.96 | α-Humulene | — | — | — |
| 28.25 | Alloaromandrene | — | — | — |
| 28.99 | α-Amorphene | — | — | — |
| 29.10 | Germacrene D | 0.62 | — | — |
| 29.66 | α-Elemene | — | — | — |
| 29.89 | α-Muurolene | — | — | — |
| 30.05 | Germacrene A | — | — | — |
| 30.42 | Butylated Hydroxy Toluene | 15.74 | — | — |
| 30.81 | δ-Cadinene | 0.66 | — | — |
| 32.26 | Spathulenol | — | 1.26 | 0.53 |
| 32.78 | γ-Cadinene | — | — | — |
| 33.20 | Caryophyllene Oxide | — | — | — |
| 34.79 | Cadina 1,4-Diene | — | — | — |
| 34.92 | γ-Eudesmol | — | — | — |
| 35.31 | α-Cadinol | — | — | — |
| 35.45 | α-Copaene | — | — | — |
| 35.58 | β-Eudesmol | — | — | — |
| 35.80 | T-Cadinolo | — | — | — |
| 56.28 | Nonadecane | — | — | — |
| 61.56 | Octadecane | — | — | — |
| 67.79 | 4Chloro2Phenylaniline | 32.88 | — | — |
| 67.96 | Benzene1Methoxy2,3,5Trimet | 25.63 | — | — |
| 69.78 | 4,12BisHydroxymethyl | 4.29 | — | — |

TABLE 1D
P. lentiscus var. chia Composition

| | | P. lentiscus var. chia |
|---|---|---|
| | | Mastic |
| RT* | Oil Composition | DM1A |
| 6.08 | Tricyclene | — |
| 6.22 | α-Phellandrene | — |
| 6.48 | α-Pinene | 72.93 |
| 6.94 | Camphene | 0.58 |
| 7.78 | Sabinene | 0.30 |
| 7.88 | β-Pinene | 2.58 |
| 8.40 | β-Myrcene | 13.57 |
| 8.86 | 1-Phellandrene | — |
| 9.02 | Methylanisol | 0.58 |
| 9.07 | δ-Carene | — |
| 9.33 | α-Terpinene | — |
| 9.69 | Para Cymene | — |
| 9.81 | β-Phellandrene | — |
| 9.89 | Limonene | 0.89 |
| 10.21 | Cis-Ocimene | — |
| 10.62 | Trans-β-Ocimene | — |
| 10.96 | Butanoic Acid | — |
| 11.03 | γ-Terpinene | — |
| 12.26 | α-Terpinolene | — |
| 12.49 | 2-Nonanone | — |
| 12.60 | α-Pinene Oxide | 0.56 |
| 12.80 | Linalool | 0.73 |
| 12.96 | Pinocarveol | 0.21 |
| 14.64 | Pinocarvone | 0.10 |
| 14.70 | Camphor | — |
| 15.74 | Endoborneol | — |
| 16.20 | 1-4 Terpineol | — |
| 16.79 | α-Terpineol | — |
| 17.48 | Myrtenol | 0.18 |
| 19.44 | Octanoid Acid | — |
| 21.04 | Bornyl Acetate | — |
| 21.39 | Verbenone | 0.26 |
| 21.42 | 2-Undecanone | — |
| 24.77 | α-Ylangene | — |
| 25.39 | β-Cubebene | — |
| 25.47 | β-Elemene | — |
| 26.61 | β-Caryophyllene | 0.30 |
| 27.85 | α-Cubebene | — |
| 27.96 | α-Humulene | — |
| 28.25 | Alloaromandrene | — |
| 28.99 | α-Amorphene | — |
| 29.10 | Germacrene D | — |
| 29.66 | α-Elemene | — |
| 29.89 | α-Muurolene | — |
| 30.05 | Germacrene A | — |
| 30.42 | Butylated Hydroxy Toluene | — |
| 30.81 | δ-Cadinene | — |
| 32.26 | Spathulenol | — |
| 32.78 | γ-Cadinene | — |
| 33.20 | Caryophyllene Oxide | — |
| 34.79 | Cadina 1,4-Diene | — |
| 34.92 | γ-Eudesmol | — |
| 35.31 | α-Cadinol | — |
| 35.45 | α-Copaene | — |
| 35.58 | β-Eudesmol | — |
| 35.80 | T-Cadinolo | — |
| 56.28 | Nonadecane | — |
| 61.56 | Octadecane | — |
| 67.79 | 4Chloro2Phenylaniline | — |
| 67.96 | BenzenelMethoxy2,3,5Trimet | — |
| 69.78 | 4,12BisHydroxymethyl | — |

RT = Retention time

Indications regarding the Pistacia species, the parts of the plant and the place of origin of the plant from which the essential oil samples used in the above experiments are given in Table 2.

TABLE 2
Synoptic table of essential oils

| | | PARTS | |
|---|---|---|---|
| ESSENTIAL | PISTACIA | OF THE | PLACE OF |
| OIL | SPECIES | PLANTS | ORIGIN |
| Portuguese | P. lentiscus | Leaves | Portugal |
| Oil | | | |
| DM1C | P. lentiscus | Leaves | Tuscany (Italy) |
| DM2A1 | P. lentiscus | Leaves | Tuscany (Italy) |
| DM3Z | P. lentiscus | Leaves | Tuscany (Italy) |
| DMF1 | P. lentiscus | Leaves | Tuscany (Italy) |
| DM3P1 | P. lentiscus | Leaves | Tuscany (Italy) |
| DMP | P. lentiscus | Berries | Tuscany (Italy) |
| DMG2 | P. lentiscus | Berries | Tuscany (Italy) |
| DM3P | P. lentiscus | Berries | Tuscany (Italy) |
| DM72 | P. lentiscus | Branches | Tuscany (Italy) |
| DM2Z | P. lentiscus | Branches | Tuscany (Italy) |
| DM1Z | P. terebinthus | Leaves | Venetia (Italy) |
| DM2S | P. terebinthus | Leaves | Venetia (Italy) |
| DMF3 | P. terebinthus | Leaves | Venetia (Italy) |
| DMV2X | P. terebinthus | Leaves | Venetia (Italy) |
| DMV5X | P. terebinthus | Leaves | Venetia (Italy) |
| DM4a | P. terebinthus | Leaves | Venetia (Italy) |
| DM4p | P. terebinthus | Leaves | Venetia (Italy) |
| DM5a | P. terebinthus | Leaves | Venetia (Italy) |
| DM4C | P. terebinthus | Leaves | Venetia (Italy) |
| DMK | P. terebinthus | Fruits | Venetia (Italy) |
| DMF2 | P. terebinthus | Fruits | Venetia (Italy) |
| DMG1 | P. terebinthus | Galls | Venetia (Italy) |
| DM1P | P. terebinthus | Galls | Venetia (Italy) |
| DM2C | P. terebinthus | Flowers | Venetia (Italy) |
| DM3C | P. terebinthus | Flowers | Venetia (Italy) |
| DM3A | P. terebinthus | Flowers | Provence (France) |
| DM2P | P. terebinthus | Branches | Venetia (Italy) |
| DM1S | P. vera | Nuts | Sicily (Italy) |
| DMNP | P. integerrima | Galls | Nepal |
| DMFt2 | P. integerrima | Galls | Nepal |
| DM1A | P. lentiscus var. chia | Mastic | Chios (Greece) |

### RESULTS OF THE BIOLOGICAL TESTS

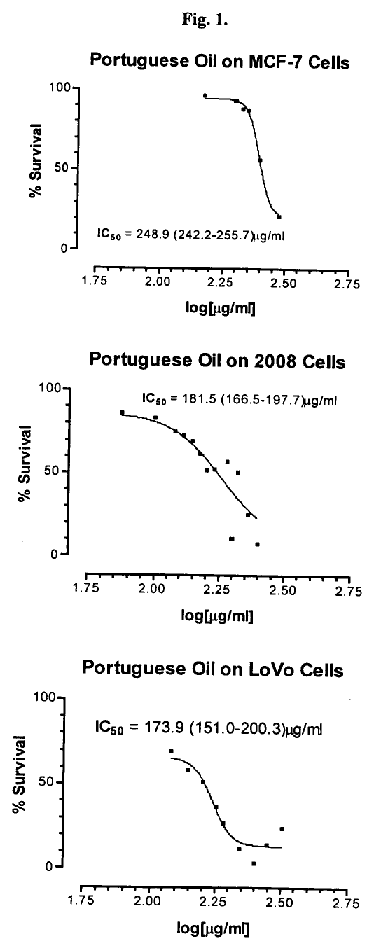

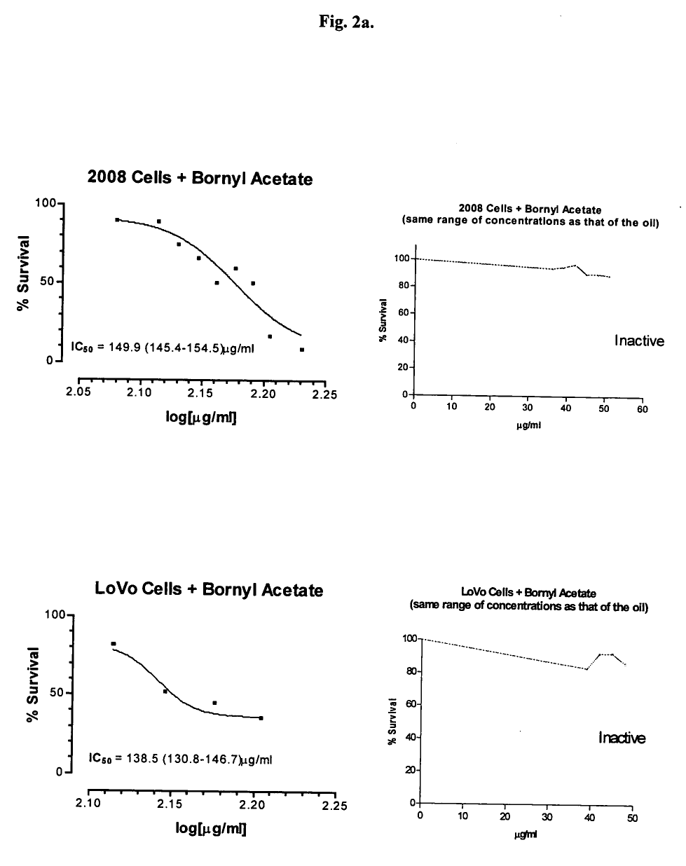

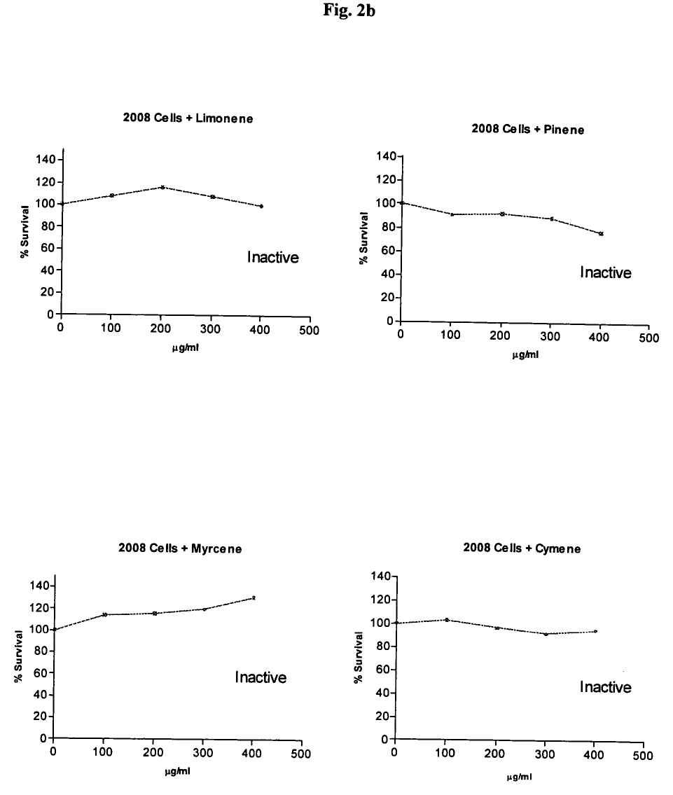

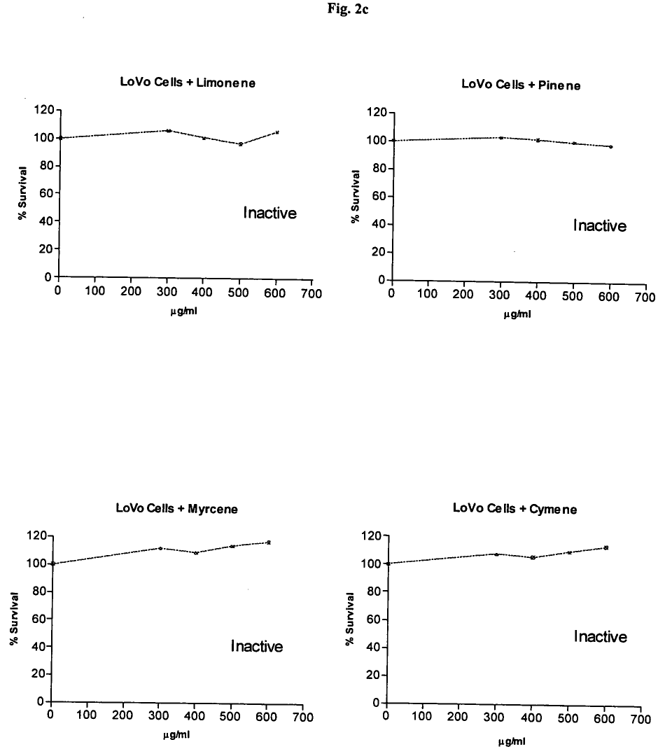

Following are some examples of results obtained evaluating the cytotoxicity of several essential oils against the selected tumor cell lines (MCF-7, LoVo and 2008) and showed that the oil from leaves of Portuguese P lentiscus (see Table 1a for composition) was active in inducing a cytotoxic effect (FIG. 1) with IC₅₀ of 248 (242.3-255.7) μg/ml on MCF-7, 181.5 (166.5-197.7) μg/ml on 2008 and 173.9 (151.0-200.3) μg/ml on LoVo cells. The cytotoxic effect was also assayed using some of the single components of the oil and the only active tested component, in our experimental conditions, was Bornyl Acetate (FIG. 2), but when this compound was utilized at the equivalent concentration of the oil it resulted inactive (FIG. 2). Among the single components tested, limonene, which has been reported in literature as having antitumoral effect, did not prove active in our experimental conditions.

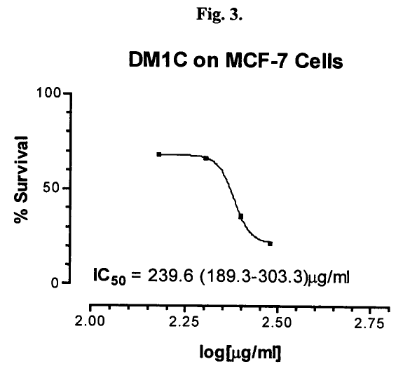

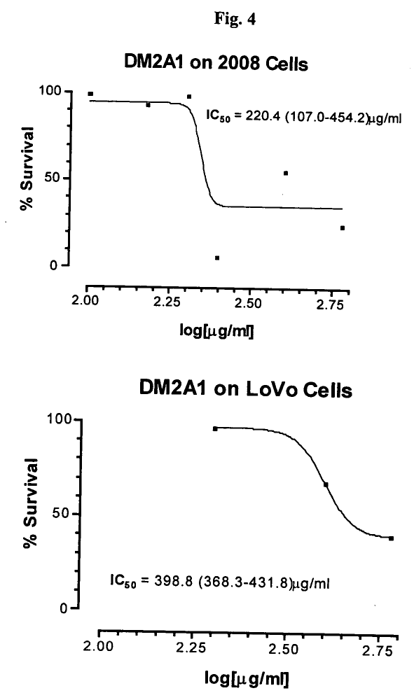

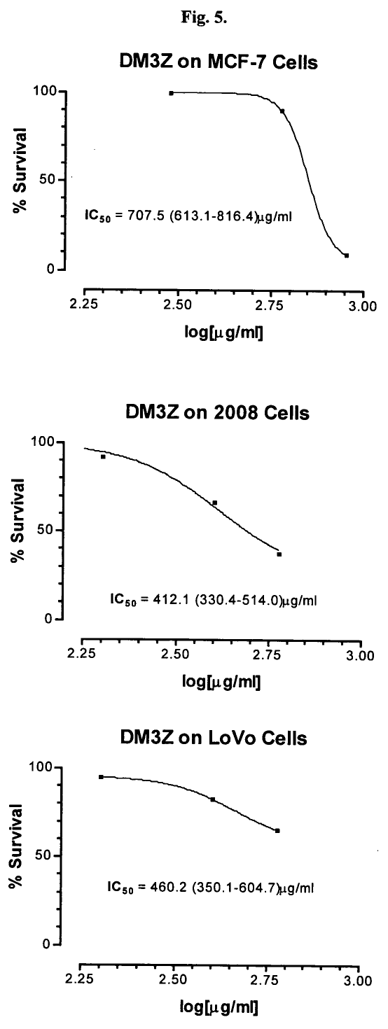

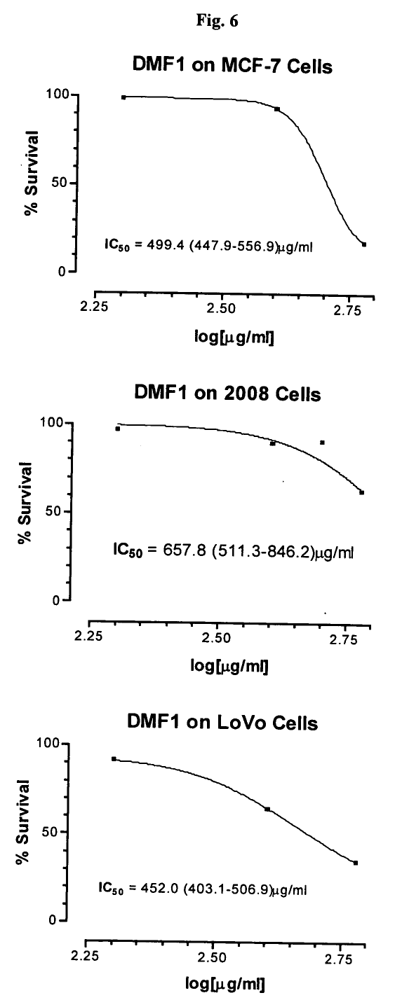

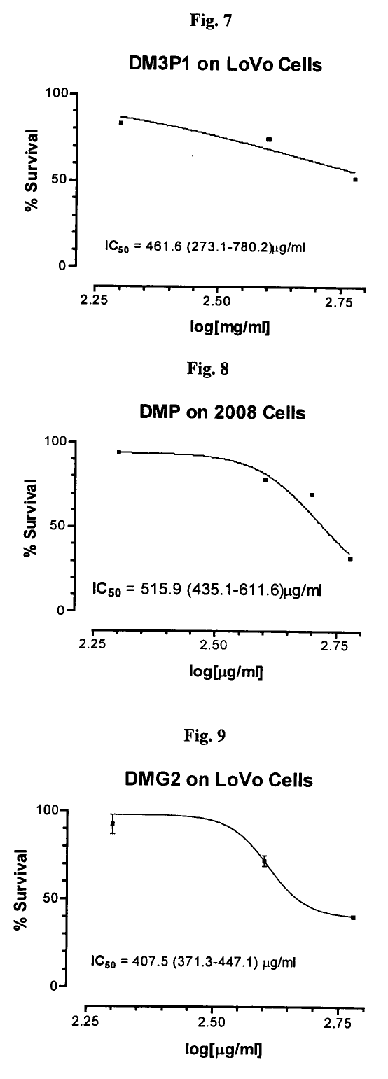

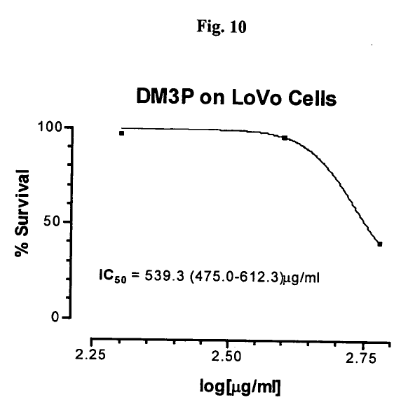

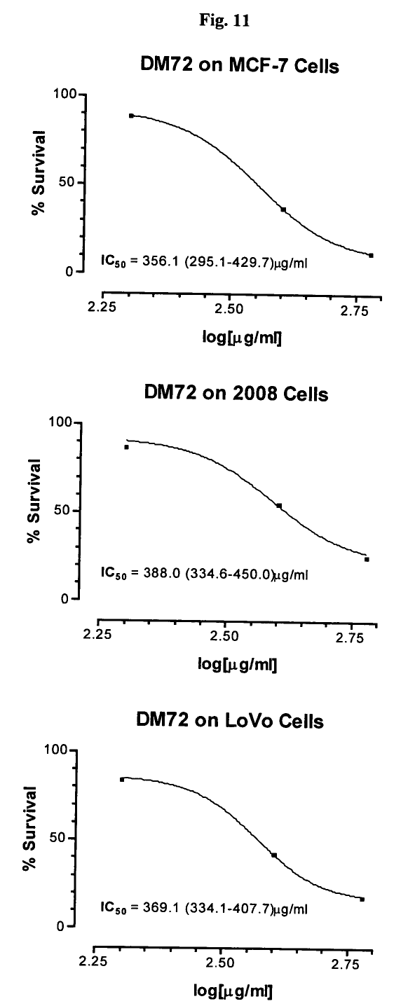

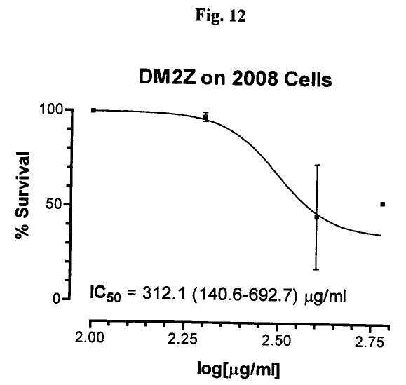

Other results showed that Pistacia essential oils were able to reduce cell growth. In particular, DM1C (oil extracted from one sample of P. lentiscus leaves) resulted active in inducing cytotoxicity in MCF-7 and LoVo cell lines with IC₅₀ of 239.6 (189.3-303.3) μg/ml (FIG. 3) and 410.8 (302.4-558.1) μg/ml, respectively. DM2A1 (oil extracted from one sample of P. lentiscus leaves) resulted active in inducing cytotoxicity in both MCF-7, 2008 and LoVo cell lines with IC₅₀ of 543.1 (503.6-585.7) μg/ml, 220.4 (107.0-454.2) μg/ml (FIG. 4) and 398.8 (368.3-431.8) μg/ml (FIG. 4), respectively. DM3Z (oil extracted from one sample of P. lentiscus leaves) resulted active in inducing cytotoxicity in both MCF-7, 2008 and LoVo cell lines with IC₅₀ of 707.5 (613.1-816.4) μg/ml, 412.1 (330.4-514.0) μg/ml and 460.2 (350.1-604.7) μg/ml, respectively (FIG. 5). DMF1 (oil extracted from one sample of P. lentiscus leaves) resulted active in inducing cytotoxicity in MCF-7, 2008 and LoVo cell lines with IC₅₀ of 499.4 (447.9-556.9) μg/ml, 657.8 (511.3-846.2) μg/ml and 452.0 (403.1-506.9) μg/ml, respectively (FIG. 6). DM3P1 (oil extracted from one sample of P. lentiscus leaves) resulted active in inducing cytotoxicity in LoVo cell lines with IC₅₀ of 461.6 (273.1-780.2) μg/ml (FIG. 7). DMP (oil extracted from one sample of P. lentiscus berries) resulted active in inducing cytotoxicity in 2008 and LoVo cell lines with IC₅₀ of 515.9 (435.1-611.6) μg/ml (FIG. 8) and 438.3 (309.0-621.6) μg/ml, respectively. DMG2 (oil extracted from one sample of P. lentiscus berries) resulted active in inducing cytotoxicity in LoVo cell lines with IC₅₀ of 407.5 (371.3-447.1) μg/ml (FIG. 9). DM3P (oil extracted from one sample of P. lentiscus berries) resulted active in inducing cytotoxicity in MCF-7 and LoVo cell lines with IC₅₀ of 299.9 (260.4-345.4) μg/ml and 539.3 (475.0-612.3) μg/ml (FIG. 10), respectively. DM72 (oil extracted from one sample of P. lentiscus branches) resulted active in inducing cytotoxicity in both MCF2-7, 2008 and LoVo cell lines with IC₅₀ of 356.1 (295.1-429.7) μg/ml, 388.0 (334.6-450.0) μg/ml and 369.1 (334.1-407.7) μg/ml, respectively (FIG. 11). DM2Z (oil extracted from one sample of P. lentiscus branches) resulted active in inducing cytotoxicity in MCF-7, 2008 and LoVo cell lines with IC₅₀ of 311.6 (260.4-373.0) μg/ml, 312.1 (140.6-692.7) μg/ml (FIG. 12) and 417.7 (392.1-445.1) μg/ml, respectively.

The essential oil extract from mastic resin (gum) and derived from plant P. lentiscus var. chia (see Table 1d for composition), unlike all the other samples of essential oil extracted from Pistacia lentiscus, is resulted inactive on MCF-7, 2008 and LoVo cells in our experimental conditions (up to 600 μg/ml concentration). The lack of biological activity registered in our experimental conditions, might be related to the profound differences in the composition of the essential oil from the P. lentiscus var. chia, respect to the other lentiscus samples (table Id).

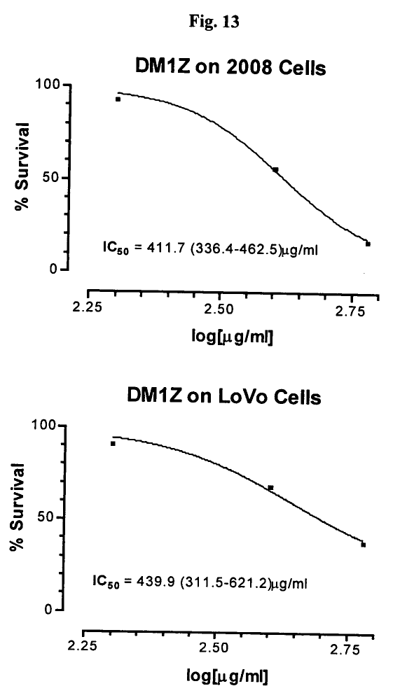

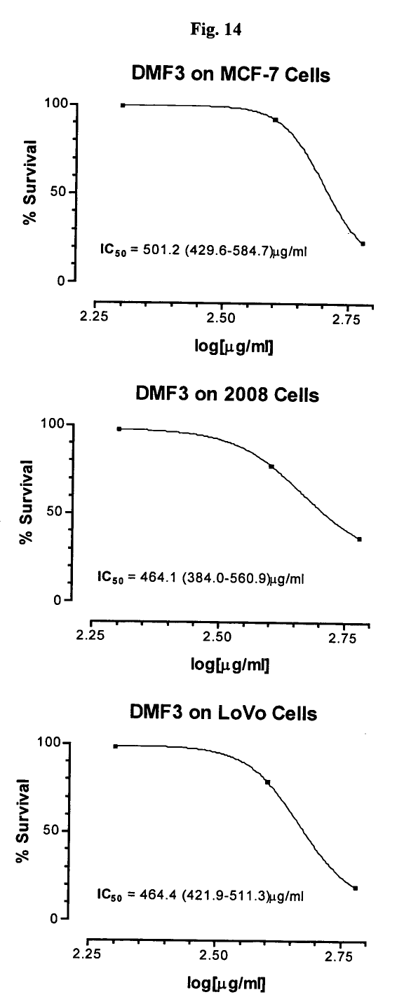

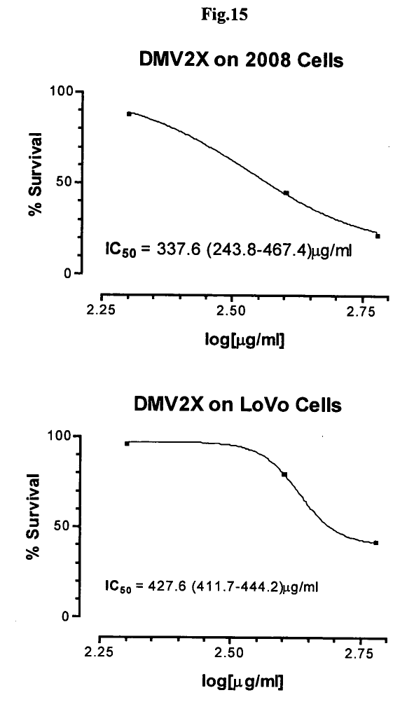

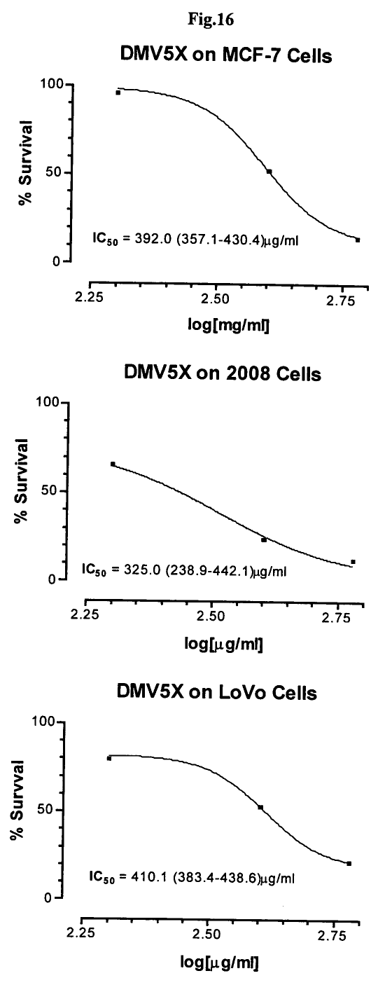

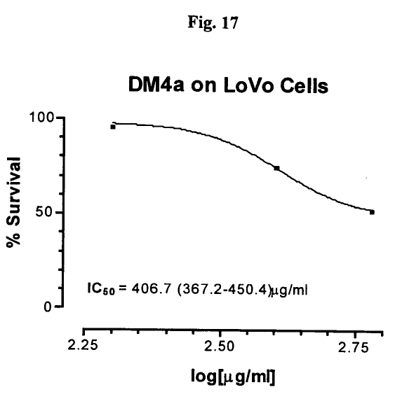

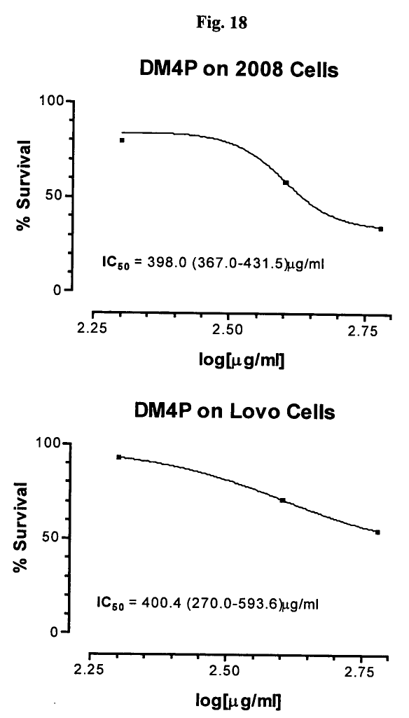

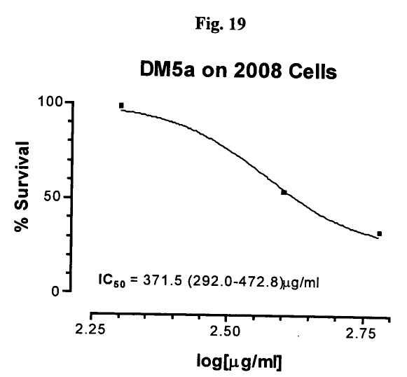

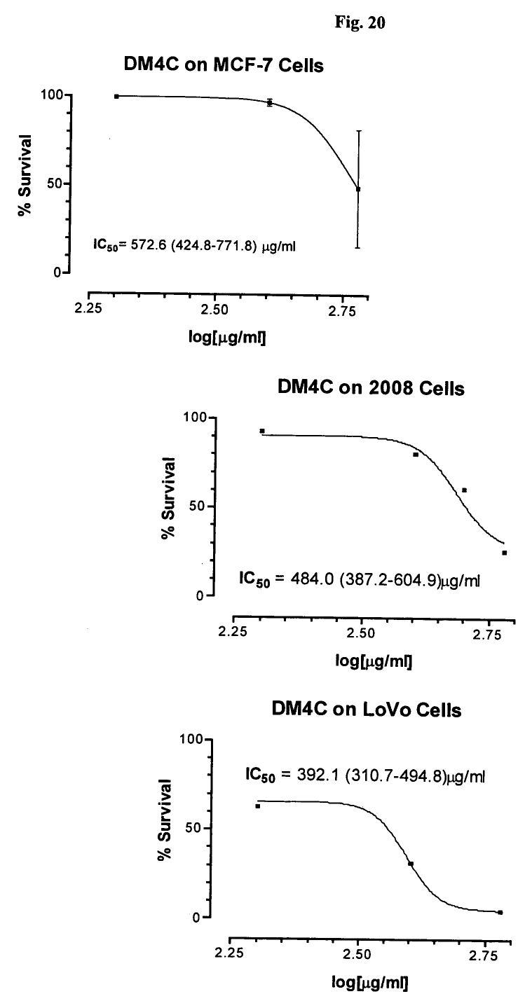

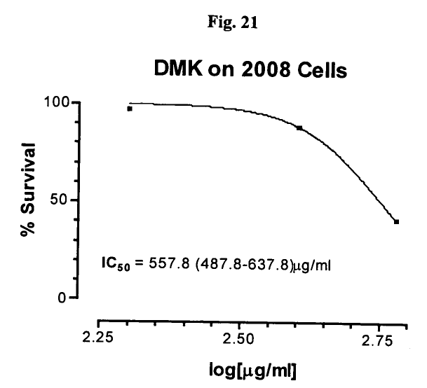

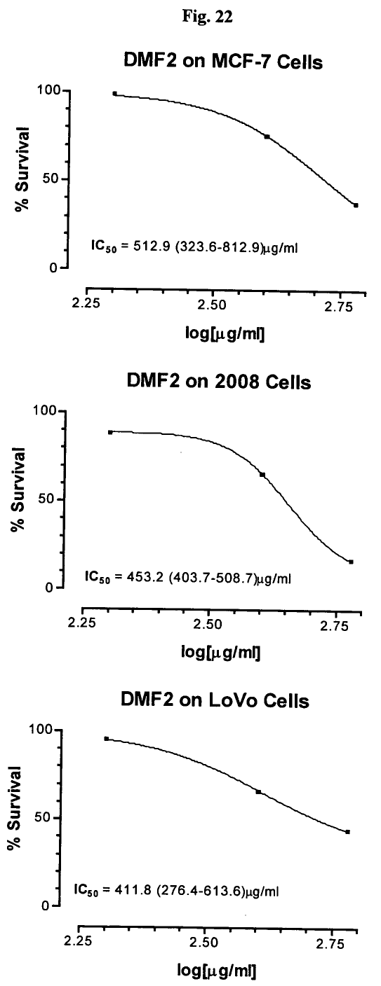

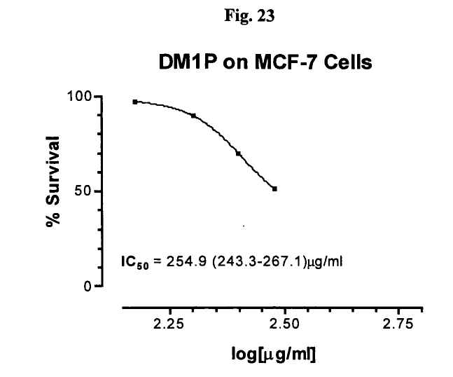

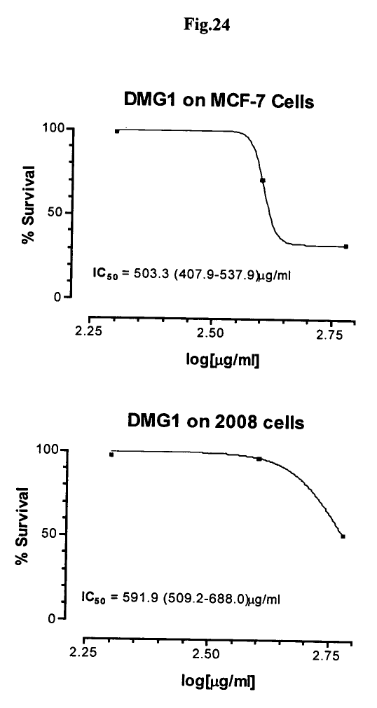

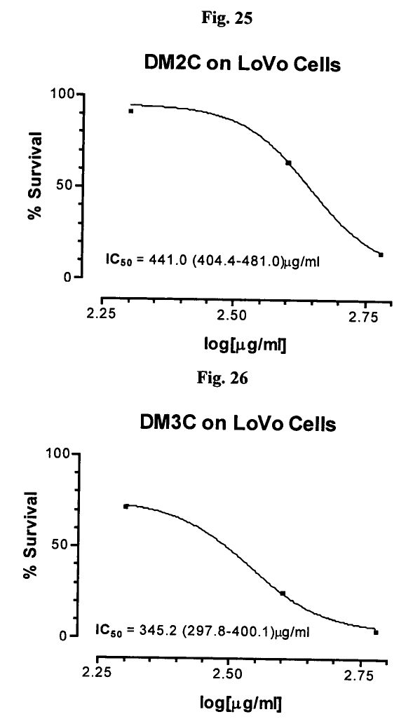

DM1Z (oil extracted from one sample of P. terebinthus leaves) resulted active in inducing cytotoxicity in both 2008 and LoVo cell lines with IC₅₀ of 411.7 (336.4-462.5) μg/ml and 439.9 (311.5-621.2) μg/ml, respectively (FIG. 13). DM2S (oil extracted from one sample of P. terebinthus leaves) resulted active in inducing cytotoxicity in LoVo cell line with IC₅₀ of 473.4 (418.8-535.1) μg/ml DMF3 (oil extracted from one sample of P. terebinthus leaves) resulted active in inducing cytotoxicity in both MCF-7, 2008 and LoVo cell lines with IC₅₀ of 501.2. (429.6-584.7) μg/ml, 464.1 (384.0-560.9) μg/ml and 464.4 (421.9-511.3) μg/ml, respectively (FIG. 14). DMV2X (oil extracted from one sample of P. terebinthus leaves) resulted active in inducing cytotoxicity in 2008 and LoVo cell lines with IC₅₀ of 337.6 (243.8-467.4) μg/ml and 427.6 (411.7-444.2) μg/ml (FIG. 15). DMV5× (oil extracted from one sample of P. terebinthus leaves) resulted active in inducing cytotoxicity in both MCF-7, 2008 and LoVo cell lines with IC₅₀ of 392.0 (357.1-430.4) μg/ml, 325.0 (238.9-442.1) μg/ml and 410.1 (383.4-438.6) μg/ml, respectively (FIG. 16). DM4a (oil extracted from one sample of P. terebinthus leaves) resulted active in inducing cytotoxicity in 2008 and LoVo cell lines with IC₅₀ of 348.1 (263.7-459.5) μg/ml and 406.7 (367.2-450.4) μg/ml (FIG. 17), respectively. DM4P (oil extracted from one sample of P. terebinthus leaves) resulted active in inducing cytotoxicity in both 2008 and LoVo cell lines with IC₅₀ of 398.0 (367.0-431.5) μg/ml and 400.4 (270.0-593.6) μg/ml, respectively (FIG. 18). DM5a (oil extracted from one sample of P. terebinthus leaves) resulted active in inducing cytotoxicity in 2008 cell lines with IC₅₀ of 371.5 (292.0-472.8) μg/ml (FIG. 19). DM4C (oil extracted from one sample of P. terebinthus leaves) resulted active in inducing cytotoxicity in both MCF-7, 2008 and LoVo cell lines with IC₅₀ of 572.6 (424.8-771.8) μg/ml, 484.0 (387.2-604.9) μg/ml and 392.1 (310.7-494.8) μg/ml, respectively (FIG. 20). DMK (oil extracted from one sample of P. terebinthus berries) resulted active in inducing cytotoxicity in 2008 and LoVo cell lines with IC₅₀ of 557.8 (487.8-637.8) μg/ml (FIG. 21) and 509.8 (377.6-688.5) μg/ml, respectively. DMF2 (oil extracted from one sample of P. terebinthus berries) resulted active in inducing cytotoxicity in MCF-7, 2008 and LoVo cell lines with IC₅₀ of 512.9 (323.6-812.9) μg/ml, 453.2 (403.7-508.7) μg/ml and 411.8 (276.4-613.6) μg/ml, respectively (FIG. 22). DMIP (oil extracted from one sample of P. terebinthus galls) resulted active in inducing cytotoxicity in MCF-7 cell lines with IC₅₀ of 254.9 (243.3-267.1) μg/ml (FIG. 23). DMG1 (oil extracted from one sample of P. terebinthus galls) resulted active in inducing cytotoxicity in MCF-7 and 2008 cell lines with IC₅₀ of 503.3 (470.9-537.9) μg/ml and 591.9 (509.2-688.0) μg/ml, respectively (FIG. 24). DM2C (oil extracted from one sample of P. terebinthus flowers) resulted active in inducing cytotoxicity in MCF-7 and LoVo cell lines with IC₅₀ of 297.7 (277.6-319.2) μg/ml and 441.0 (404.4-481.0) μg/ml (FIG. 25), respectively. DM3C (oil extracted from one sample of P. terebinthus flowers) resulted active in inducing cytotoxicity in LoVo cell lines with IC₅₀ of 345.2 (297.8-400.1) μg/ml (FIG. 26). DM3A (oil extracted by P. terebinthus flowers from Provence in France) resulted active in inducing cytotoxicity in 2008 and LoVo cell lines with IC₅₀ of 331.4 (266.9-411.5) μg/ml and 623.1 (554.8-699.8) μg/ml, respectively

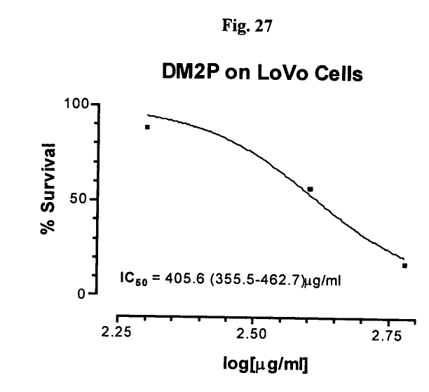

DM2P (oil extracted from one sample of P. terebinthus branches) resulted active in inducing cytotoxicity in LoVo cell lines with IC₅₀ of 405.6 (355.5-462.7) μg/ml (FIG. 27).

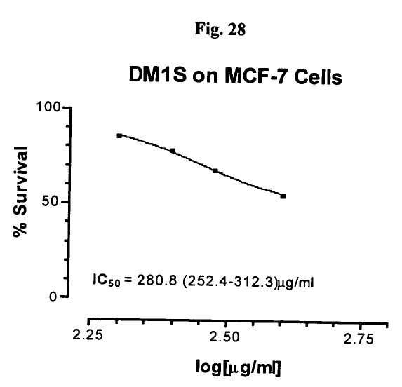

DM1S (oil extracted from one sample of P. vera nuts) resulted active in inducing cytotoxicity in MCF-7 cell lines with IC₅₀ of 280.8 (252.4-312.3) μg/ml (FIG. 28).

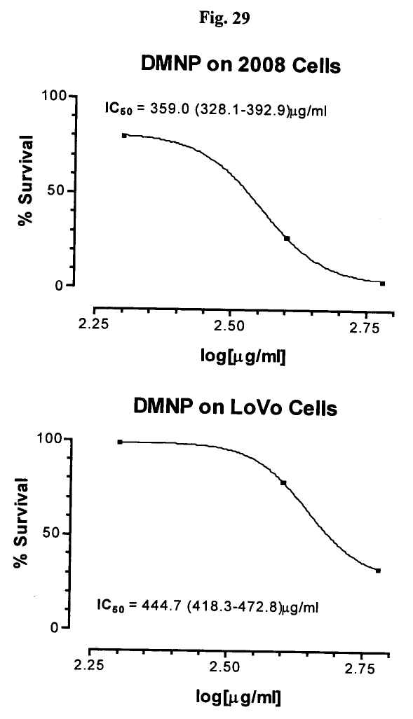

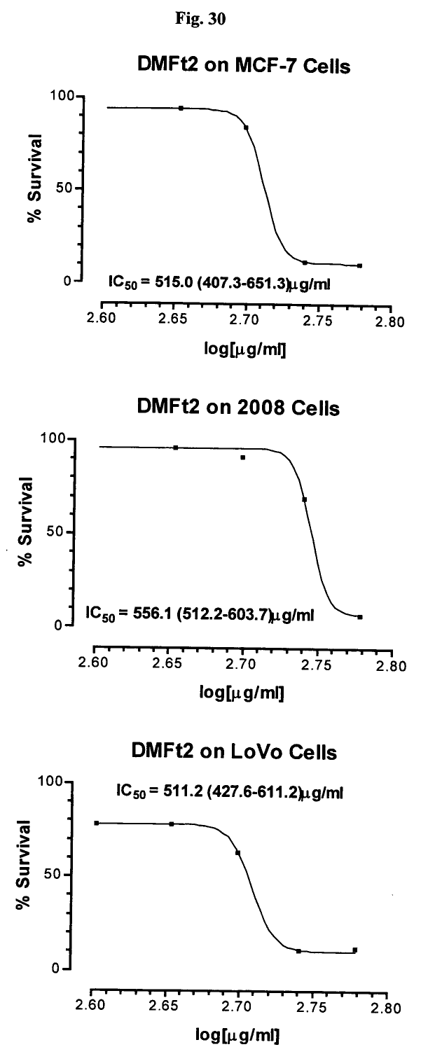

DMNP (oil extracted from one sample of P. integerrima galls) resulted active in inducing cytotoxicity in both 2008 and LoVo cell lines with IC₅₀ of 359.0 (328.1-392.9) μg/ml and 444.7 (418.3-472.8) μg/ml, respectively (FIG. 29). DMFt2 (oil extracted from one sample of P. integerrima galls) resulted active in inducing cytotoxicity in MCF-7, 2008 and LoVo cell lines with IC₅₀ of 515.0 (407.3-651.3) μg/ml, 556.1 (512.2-603.7) μg/ml and 511.2 (427.6-611.2) μg/ml, respectively (FIG. 30).

The activities of DM2A1, DM1Z, DM3Z, as well as that of the oil from Portugal were also tested with two cytofluorimetric assays to analyse the nature of cell death (Annexin V plus Propidium Iodide; NO production), which resulted mostly in apoptotic death for all oils.

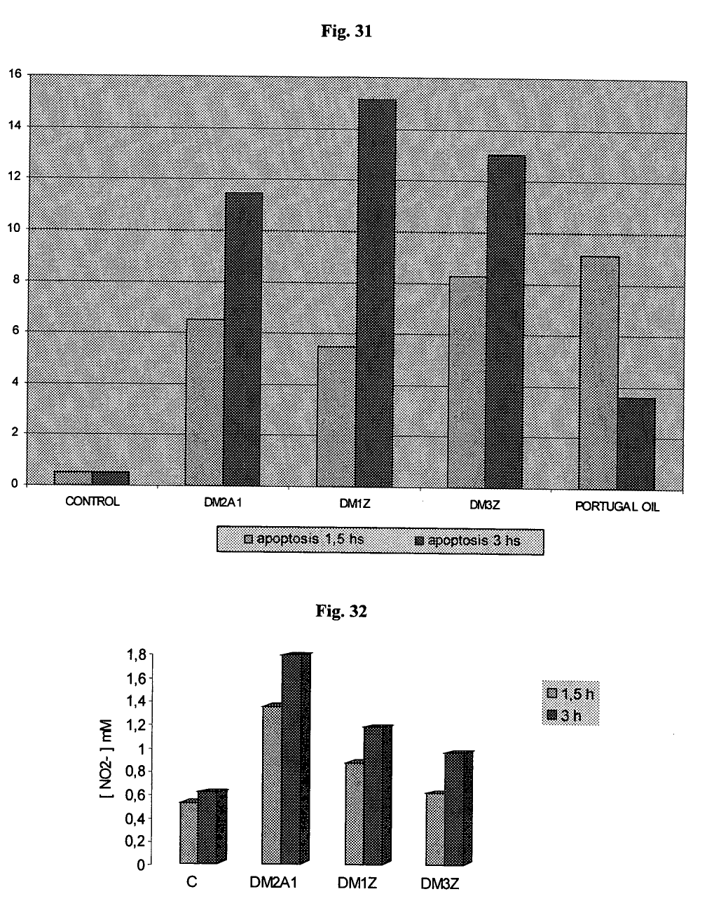

The cytofluometric tests indicated a cytotoxic effect with the same oils. FIG. 31-32

The results obtained evaluating the nitrite concentration in the culture medium of 2008 cells treated for 1.5 and 3 h with DM2A1, DM1Z and DM3Z indicated the activation of apoptotic mechanisms. It should be pointed out that these results were in accordance also to the cytotoxicity studies, indeed increasing the NO detected in medium, increasing the cytotoxic effect: DM2A1 was the most cytotoxic oil on 2008 cells and was able to induce the most production of NO.

Correlation Analysis

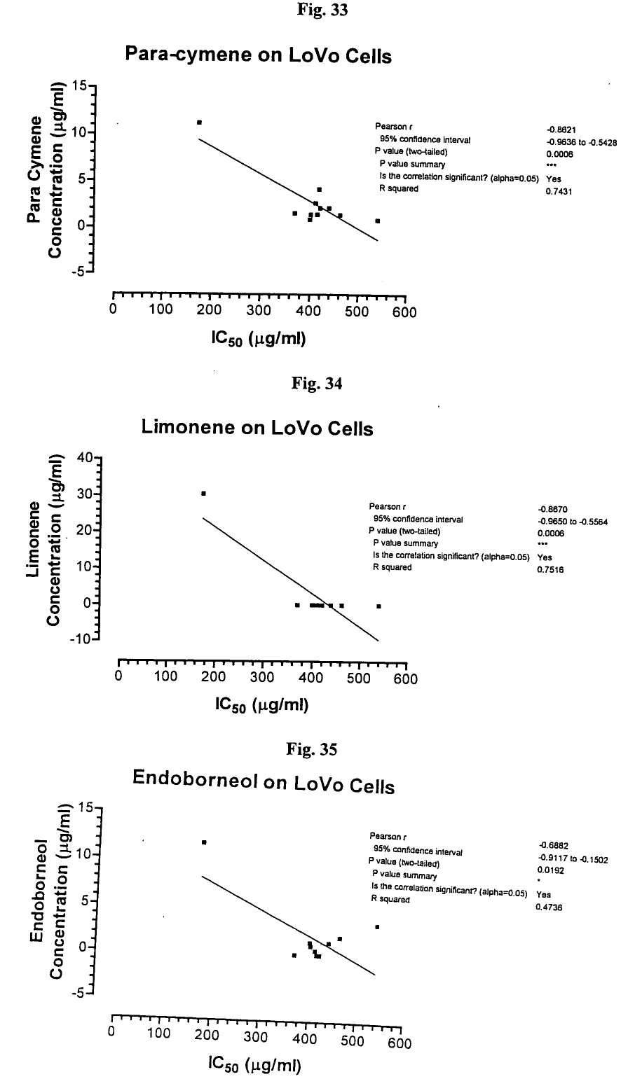

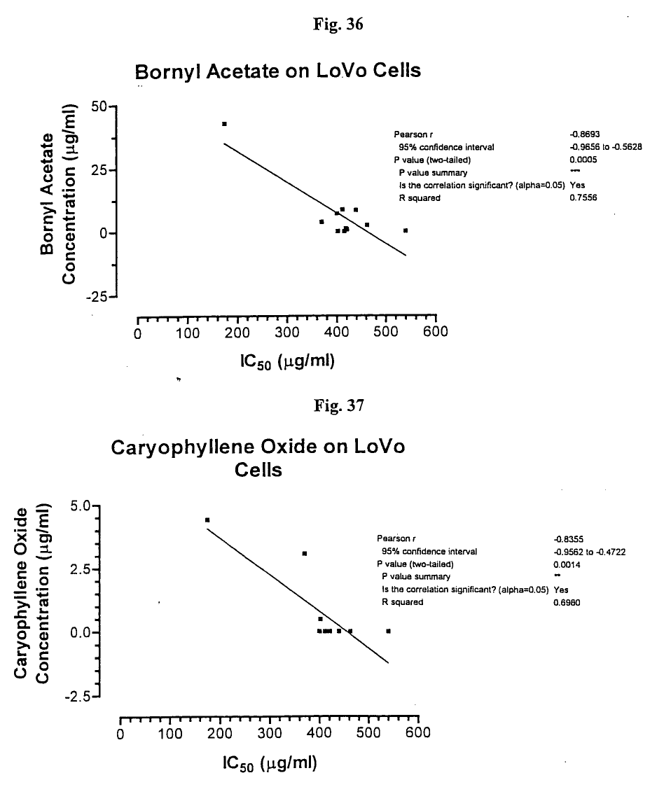

The analysis to identify possible correlation between oil components and cytotoxic biological activity resulted in the identification of several positive correlations, but only 5 oil components were significantly correlated with the activity: Para cymene (P. lentiscus/LoVo cells: r=−0.8621, p value=0.0006) (FIG. 33); Limonene (P. lentiscus/LoVo cells: r=0.8670, p value=0.0006) (FIG. 34); Endoborneol (P. lentiscus/LoVo cells: r=0.6882, p value=0.0192) (FIG. 35); Bornyl Acetate (P. lentiscus/LoVo cells: r=0.8693, p value=0.0005) (FIG. 36); Caryophyllene Oxide (P. lentiscus/LoVo cells: r=0.8355, p value=0.0014) (FIG. 37).

### REFERENCES

Boelens M. H., Jemenez R. Chemical composition of the essential oil from the gum and various parts of Pistacia lentiscus L. (Mastic Gum Tree). Flav.Fragr.J., 6:271-275,1991.

Buffoni F. La natura come fonte inesauribile di farrnaci. Acta Phytotherapeutica., 2,3-6,1996.

Buil P. et al. Contribution à la connaissance de la composition chimique de l'essence de lentisque de Provence. Riv. Ital. EPPOS Cosmet. Aerosol., 56:245-252,1975.

Calabro G., Curro P. Costituenti degli oli essenziali Nota IV. Essenza di lentisco. Essence Deriv.Agrum., 44:82-92,1974.

Castola V. et al. Intraspecific chemical variabilita of the essential oil of Pistacia lentiscusL. from Corsica. Biochem. System. Ecol., 28:79-88,2000.

Chander S. K., Lansdown A. G. B., Luqmani Y. A., Gomm J. J., Coope R. C., Gould M. N. & Coope R. C. Gould M. N. & Coombes R. C. Effectiveness of combined limonene and 4-hydroxyandrostenedione in the treatment of NMU-induced rat mammary tumors. Br. J. Cancer., 69:879-882.

Colombini M. P. et al. Characterization of the balm of an Egyptian mummy from the seventh century B.C. Studies in Conservation., 45:19-29,2000.

Conner D. E., Beuchat L. R. Sensitivity of heat stressed yeasts to essential oils of plants. Appl. Environ. Microbiol., 47:229-233,1984.

Cragg G. M. et al. Natural products in drug discovery and development. J. Nat. Prod, 60:52-60,1997.

Cragg G. M., Newman D. J. Discovery and development of antineoplastic agents from natural sources. Cancer Invest., 17:153-163,1999.

Croteau R. Biosynthesis and catabolism of monoterpenoids. Chem. Rev., 87:929-954,1987.

Crowell P. L., Kennan W. S., Haag J. D., Ahmad S., Vedejs E. & Gould M. N. Chemoprevention of mammary carcinogenesis by hydroxylated derivatives of d-limonene. Carcinogenesis., 13:1261-1264,1992.

Crowell P. L., Elson, Bailey, H. H., C. E., Elegbede A., Haag J. H., Gould M. N. Human metabolism of the experimental cancer therapeutic agent d-limonene. Cancer Chemother. Pharmacol., 35:31-37,1994 (a).

Crowell P. L., Gould M. N. Chemoprevention and therapy of cancer by d-limonene. CRC. Crit. Rev. Oncogenesis., 5:1-22,1994 (b).

Crowell P. L. et al. Dietary Phytochemicals in Cancer Prevention and Treatment. Plenum Press, New York, 1996,

Crowell P. L. Prevention and therapy of cancer by dietary monoterpenes. J. Nutr., 129(3):775S-778S,1999.

De Pooter H. L. et al. Essential oil of the leaves of three Pistacia species grown in Egypt. Flav. Fragr. J. 6:229-232,1991.

Dietrich D. R. & Swenberg J. A. The presence of α₂ᵤ-globulin is necessary for d-limonene promotion of male rat kidney tumors. Cancer Res., 51:3512-3517,1991.

Elegbede J. A., Elson C. E., Qureshi A., Tanner M. A. & Gould M. N. Inhibition of DMBA-induced mammary cancer by the monoterpene d-limonene. Carcinogenesis., 5:661-665,1984.

Elegbede J. A., Maltzman T. H., Verma A. K., Tanner M. A. & Gould M. N. Mouse skin tumor promoting activity of orange peel oil and d-limonene: a reevaluation. Carcinogenesis., 7:2047-2049,1986 (a).

Elegbede J. A., Elson C. E., Tanner M. A., Qureshi A. & Gould M. N, Regression of rat primary mammary tumors following dietary d-limonene. J. Natl. Cancer Inst., 76:323-325,1986 (b).

Elson C. E., Maltzman T. H., Boston J. L., Tanner M. A. & Gould M. N. Anti-carcinogenic activity of d-limonene during the initiation and promotion-progression stages of DMBA-induced rat mammary carcinogenesis. Carcinogenesis., 9:331-332,1988.

Elson C. E., Yu S. G. The chemoprevention of cancer by mevalonate-derived constituents of fruits and vegetables. J. Nutr., 124:607-614,1994.

Elson C. E. Suppression of mevalonate pathway activities by dietary isoprenoids: protective roles in cancer roles in cancer and cardiovascuar disease. J. Nutr., 125:1666S-1672S,1995.

Evans E., Arneson D., Kovatch R., Supko J., Morton T., Siemann L., Cannon R., Tomaszewski J., Smith A. Toxicology and pharmacology of perillyl alcohol (NSC-641066) in rats and dogs. Proc. Am. Assoc. Cancer Res., 36:366,1995.

Farnsworth N. R. et al. Medicinal plants in therapy. Bull World Health Organ., 63:965-981,1985.

Farnsworth N. R. The role of ethnopharmacology in drug development. Ciba Found Symp., 154:2-11,1990.

Giner-Larza Eva m. el al. On the anti-inflammatory and anti-phospholipase A₂ activity of extract from lanostane-rich species. ETH. J, 73:61-69,2000.

Gould M. N., Moore C. J., Zhang R., Wang B., Kennan W. S. & Haag J. D. Limonene chemoprevention of mammary carcinoma induction following direct in situ transfer of v-Ha-ras. Cancer Res., 54:3540-3543,1994.

Haag J. D., Lindstrom M. J. & Gould M. N. Limonene-induced regression of mammary carcinomas. Cancer Res., 52:4021-4026,1992.

Haag J. D. & Gould M. N. Mammary carcinoma regression induced by perillyl alcohol, a hydroxylated analog of limonene. Cancer. Chemother. Pharmacol., 34;477-483,1994.

Hursting S. D., Perkins S. N., Haines D. C., Ward J. M.& Phang J. M. Chemoprevention of spontaneous tumorigenesis in p53-knockout mice. Cancer Res., 55:3949-3953, 1995.

Huwez F. U. et al. Mastic Gum Kills Helicobacter pylori. N. Engl. J. Med., 339(26):365,1998.

Jeffers L., Church D., Gould M. and Wilding G. The effect of perillyl alcohol on the proliferation of human prostatic cell lines. Proc. Am. Assoc. Cancer Res., 36:303, 1995.

Jirtle R. L., Haag J: D., Ariazi E., Gould M. N. Increased mannose 6-phosphate/insulin-like growth factor II receptor and transforming growth fact b 1 levels during monoterpene-induced regression of mammary tumors. Cancer Res., 53:3849-3853, 1993.

Kawamori T., Tanaka T., Hirose Y., Ohnishi M. & Mori H. Inhibitory effects of d-limonene on the development of colonic aberrant crypt foci induced by azoxymethane in F344 rats. Carcinogenesis., 17:369-372,1996.

Kawata S., Nagase T., Yamasaki E., Ishiguro H., Matsuzwawa Y. Modulation of the mevalonate pathway and cell growth by pravastatin and d-limonene in a human hepatoma cell line (Hep G2). Br. J. Cancer., 69:1015-1020,1994.

Magiatis P. et al. Chemical Composition and Antimicrobial Activity of the Essential Oils of Pistacia lentiscus var.chia. Planta Med., 65(8):749-752,1999.

Maltzman T. H., Hurt L. M., Elson C.E., Tanner M. A. & Gould M. N. The prevention of nitrosomethylurea-induced mammary tumors by d-limonene and orange oil. Carcinogenesis., 10:781-785,1989.

Marone P. et al. Bacterial activity of Pistacia lentiscus mastic gum against Helicobacter pylori. J. Chemoth., 13(6):611-614,2001.

McNamee D. Limonene trial in cancer. Lancet., 342:801,1993.

Mills J. J., Chari R. S., Boyer I. J., Gould M. N. & Jirtle R. L. Induction of apoptosis in liver tumors by the monoterpene perillyl alcohol. Cancer Res., 55:979-983,1995.

Papageorgiou V. P. et al. GLC-MS computer analysis of the essential oil of mastic gum. Chim. Chronica, New Ser., 10:119-124,1981.

Phillips L. R., Malspeis L. & Supko J. G. Pharmnacokinetics of active drug metabolites after oral administration of perillyl alcohol, an investigational antineoplastic agent, to the dog. Drug Metab. Dispos., 23:676-680.

Ray M., Kratz D., Lewis K., Hohl R. J. Effects of combinations of lovastatin and monoterpenes on ras processing. Proc of AACR., 36:428,1995.

Ren Z. and Gould M. N. Inhibition of ubiquinone and cholesterol synthesis by the monoterpene perillyl alcohol. Cancer Lett. 76:185-190, 1994.

Rodbard D and Frazier G R. Statistical analysis of radioligand assay data. Methods Enzymol. 37:3-22;1975.

Russin W. A., Hoesly J. D., Elson C. E., Tanner M. A. & Gould M. N. Inhibition of rat mammary carcinogenesis by monoterpenoids. Carcinogenesis., 10:2161-2165,1989.

Sanz M. J. et al. In vivo hypoiensive activity of Pistacia lentiscus L. Phytother. Res., 2(4):201-203,1988.

Scurbis B., Markakis P. Essential oil of mastic gum. Int. Flavours Food Addit., 6:349,1975.

Shoff S. M., Grummer M., Yatvin M. B. & Elson C. E. Concentration-dependent increase of murine P388 and B16 population doubling time by the acyclic monoterpene geraniol. Cancer Res., 51:37-42,1991.

Stark M. J. et al. Chemotherapy of pancreatic cancer with the monoterpene perillyl alcohol. Cancer lett., 96(1):15-21,1995.

Wattenberg L. W. Inhibition of neoplasia by minor dietary constituents. Cancer Res., 43:2448s-2453s,1983.

Wattenberg L. W., Sparnins V. L. & Barany G. Inhibition of N-nitrosodiethylamine carcinogenesis in mice by naturally occurring organosulfur compounds and monoterpenes. Cancer Res., 49:2689-2694,1989.

Wattenberg L. W., & Coccia J. B. Inhibition of 4-(methylnitrosoamino)-1-butanone carcinogenesis in mice by d-limonene and citrus fruit oils. Carcinogenesis., 12:115-117,1991.

Wyllie S. G. et al. Volatile components of the fruits of Pistacia lentiscus. J. Food Sci., 55:1325-1326,1990.

Yu S. G., Hildebrandt L. A. & Elson C. E. Geraniol, an inhibitors of mevalonate biosynthesis, suppresses the growth of hepatomas and melanomas transplanted to rats and mice. J. Nutr., 125:2763-2767,1995.

1. A method for killing tumoral cells comprising exposing the tumoral cells to an effective amount of a product obtainable from a plant of Pistacia genus, provided that the plant is not p. Lentiscus var. chia.

2. The method of claim 1 wherein the tumoral cells are selected from the group consisting of breast adenocarcinoma cells, colon adenocarcinoma cells and ovary adenocarcinoma cells.

3. The method of claim 1, wherein the plants of the Pistacia genus are selected from the group consisting of p. Terebinthus, p. Lentiscus, p. Vera and p. Integerrima.

4. The method of claim 3, wherein the plant of the Pistacia genus is p. Lentiscus.

5. The method of claim 4 wherein the tumoral cells are selected from the group consisting of breast adenocarcinoma cells, colon adenocarcinoma cells and ovary adenocarcinoma cells.

6. The method of claim 4, wherein the plant of the Pistacia genus is p. Lentiscus of European origin.

7. The method of claim 6 wherein the tumoral cells are selected from the group consisting of breast adenocarcinoma cells, colon adenocarcinoma cells and ovary adenocarcinoma cells.

8. The method of claim 4, wherein the plant of the Pistacia genus is p. Lentiscus of Iberic or Italian origin.

9. The method of claim 8 wherein the tumoral cells are selected from the group consisting of breast adenocarcinoma cells, colon adenocarcinoma cells and ovary adenocarcinoma cells.

10. The method of claim 3, wherein the plant of the Pistacia genus is p. Terebintus.

11. The method of claim 10 wherein the tumoral cells are selected from the group consisting of breast adenocarcinoma cells, colon adenocarcinoma cells and ovary adenocarcinoma cells.

12. The method of claim 10, wherein the plant of the Pistacia genus is p. Terebintus of European origin.

13. The method of claim 12 wherein the tumoral cells are selected from the group consisting of breast adenocarcinoma cells, colon adenocarcinoma cells and ovary adenocarcinoma cells.

14. The method of claim 12, wherein the plant of the Pistacia genus is p. Terebintus of Italian or French origin.

15. The method of claim 14 wherein the tumoral cells are selected from the group consisting of breast adenocarcinoma cells, colon adenocarcinoma cells and ovary adenocarcinoma cells.

16. The method of claim 3, wherein the plant of the Pistacia genus is p. Vera.

17. The method of claim 16 wherein the tumoral cells are breast adenocarcinoma cells.

18. The method of claim 16, wherein the plant of the Pistacia genus is p. Vera of European origin.

19. The method of claim 18 wherein the tumoral cells are breast adenocarcinoma cells.

20. The method of claim 16, wherein the plant of the Pistacia genus is p. Vera of Sicilian origin.

21. The method of claim 20 wherein the tumoral cells is breast adenocarcinoma cells.

22. The method of claim 3, wherein the plant of the Pistacia genus is p. Integerrima.

23. The method of claim 23 wherein the tumoral cells are selected from the group consisting of breast adenocarcinoma cells, colon adenocarcinoma cells and ovary adenocarcinoma cells.

24. The method of claim 1 wherein the product is (-)-bornyl acetate.

25. The method of claim 24 wherein the tumoral cells are selected from the group consisting of breast adenocarcinoma cells, colon adenocarcinoma cells and ovary adenocarcinoma cells.

26. The method of claim 1 wherein the product is a natural or synthetic derivative of (-)-bornyl acetate.

27. The method of claim 26 wherein the tumoral cells are selected from the group consisting of breast adenocarcinoma cells, colon adenocarcinoma cells and ovary adenocarcinoma cells.

28. The method of claim 1 wherein the product is an essential oil.

29. The method of claim 28 wherein the tumoral cells are selected from the group consisting of breast adenocarcinoma cells, colon adenocarcinoma cells and ovary adenocarcinoma cells.

30. The method of claim 1 wherein the product is a component of an essential oil.

31. The method of claim 30 wherein the tumoral cells are selected from the group consisting of breast adenocarcinoma cells, colon adenocarcinoma cells and ovary adenocarcinoma cells.

32. A method for killing tumoral cells comprising exposing the tumoral cells to an effective amount of one or more products obtainable from plants of Pistacia genus or parts thereof, wherein the plants of the Pistacia genus are selected from the group consisting of p. Terebinthus, p. Lentiscus, p. Vera and p. Integerrima provided that the plant is not p. Lentiscus var. chia, the tumoral cells are selected from the group consisting of breast adenocarcinoma cells, colon adenocarcinoma cells and ovary adenocarcinoma cells and the one or more products are selected from essential oils, components of essential oils and mixtures and derivatives thereof.
